SEQUENCE LISTING (6 sequences)

SEQ ID NO: 1 - DNA, 1473 bp, Aspergillus terreus
  source 1..1473: mol_type = genomic DNA; note = aureus
atgaccaaacagagcgcagatagcaatgcaaaaagcggtgttaccagcgaaatttgtcattgggcaagcaatctggcaaccgatgatattccgagtgatgttctggaacgtgccaaatatctgattctggatggtattgcatgtgcatgggttggtgcacgtgttccgtggtcagaaaaatatgttcaggcaaccatgagctttgaaccgcctggtgcatgtcgtgttattggttatggccagaaactgggtccggttgcagcagcaatgaccaatagcgcatttattcaggccaccgaactggatgattaccatagcgaagcaccgctgcatagcgcaagcattgttctgcctgccgtttttgcagcaagcgaagttctggcagaacagggtaaaaccattagcggtattgatgttattctggcagccattgttggttttgaaagcggtccgcgtattggtaaagcaatttatggtagcgatctgctgaataatggttggcattgtggtgcagtttatggtgcaccggcaggcgcactggccaccggcaaactgctgggtctgacaccggatagcatggaagatgcactgggtattgcctgtacccaggcatgtggtctgatgagcgcacagtatggtggtatggttaaacgtgttcagcatggttttgcagcccgtaatggtctgctgggtggcctgctggcacatggtggttatgaagcaatgaaaggtgtgctggaacgtagctatggtggttttctgaaaatgtttaccaaaggcaatggtcgtgaacctccgtataaagaagaagaagttgttgcaggtctgggtagcttttggcatacctttaccattcgcattaaactgtatgcatgttgtggtctggttcatggtccggtggaagcaattgaaaatctgcaaggtcgttatccggaactgctgaatcgtgcaaatctgagcaatattcgtcatgttcatgttcagctgagcaccgcaagcaatagccattgcggttggattccggaagaacgtccgattagcagcattgcaggtcagatgagcgttgcatatattctggccgttcagctggttgatcagcagtgtctgctgagccagtttagcgaatttgatgataacctggaacgtccggaagtttgggatctggcacgtaaagttaccagcagccagagcgaagaatttgatcaggatggtaattgtctgagcgcaggtcgtgttcgtattgaatttaatgatggttccagcattaccgaaagcgttgaaaaaccgctgggtgttaaagaaccgatgccgaatgaacgtatcctgcataaatatcgtaccctggcaggtagcgttaccgatgaaagccgtgtgaaagaaattgaggatctggttctgggtctggatcgtctgaccgatattagtccgctgctggaactgctgaactgtccggttaaaagtccgctggtttaa

SEQ ID NO: 2 - DNA, 2829 bp, Corynebacterium glutamicum
  source 1..2829: mol_type = genomic DNA
gagctcactgtgactgaaagcaagaactccttcaatgctaagagcacccttgaagttggcgacaagtcctatgactacttcgccctctctgcagtgcctggcatggagaagctgccgtactccctcaaggttctcggagagaaccttcttcgtaccgaagacggcgcaaacatcaccaacgagcacattgaggctatcgccaactgggatgcatcttccgatccaagcatcgaaatccagttcaccccagcccgtgttctcatgcaggacttcaccggtgtcccttgtgtagttgacctcgcaaccatgcgtgaggcagttgctgcactcggtggcgaccctaacgacgtcaacccactgaacccagccgagatggtcattgaccactccgtcatcgtggaggctttcggccgcccagatgcactggctaagaacgttgagatcgagtacgagcgcaacgaggagcgttaccagttcctgcgttggggttccgagtccttctccaacttccgcgttgttcctccaggaaccggtatcgtccaccaggtcaacattgagtacttggctcgcgtcgtcttcgacaacgagggccttgcatacccagatacctgcatcggtaccgactcccacaccaccatggaaaacggcctgggcatcctgggctggggcgttggtggcattgaggctgaagcagcaatgctcggccagccagtgtccatgctgatccctcgcgttgttggcttcaagttgaccggcgagatcccagtaggcgttaccgcaactgacgttgtgctgaccatcaccgaaatgctgcgcgaccacggcgtcgtccagaagttcgttgagttctacggctccggtgttaaggctgttccactggctaaccgtgcaaccatcggcaacatgtccccagagttcggctccacctgtgcgatgttcccaatcgacgaggagaccaccaagtacctgcgcctcaccggccgcccagaagagcaggttgcactggtcgaggcttacgccaaggcgcagggcatgtggctcgacgaggacaccgttgaagctgagtactccgagtacctcgagctggacctgtccaccgttgttccttccatcgctggccctaagcgcccacaggaccgcatccttctctccgaggcaaaggagcagttccgtaaggatctgccaacctacaccgacgacgctgtttccgtagacacctccatccctgcaacccgcatggttaacgaaggtggcggacagcctgaaggcggcgtcgaagctgacaactacaacgcttcctgggctggctccggcgagtccttggctactggcgcagaaggacgtccttccaagccagtcaccgttgcatccccacagggtggcgagtacaccatcgaccacggcatggttgcaattgcatccatcacctcttgcaccaacacctctaacccatccgtgatgatcggcgctggcctgatcgcacgtaaggcagcagaaaagggcctcaagtccaagccttgggttaagaccatctgtgcaccaggttcccaggttgtcgacggctactaccagcgcgcagacctctggaaggaccttgaggccatgggcttctacctctccggcttcggctgcaccacctgtattggtaactccggcccactgccagaggaaatctccgctgcgatcaacgagcacgacctgaccgcaaccgcagttttgtccggtaaccgtaacttcgagggacgtatctcccctgacgttaagatgaactacctggcatccccaatcatggtcattgcttacgcaatcgctggcaccatggacttcgacttcgagaacgaagctcttggacaggaccaggacggcaacgacgtcttcctgaaggacatctggccttccaccgaggaaatcgaagacaccatccagcaggcaatctcccgtgagctttacgaagctgactacgcagatgtcttcaagggtgacaagcagtggcaggaactcgatgttcctaccggtgacaccttcgagtgggacgagaactccacctacatccgcaaggcaccttacttcgacggcatgcctgtcgagccagtggcagtcaccgacatccagggcgcacgcgttctggctaagctcggcgactctgtcaccaccgaccacatctcccctgcttcctccattaagccaggtacccctgcagctcagtacttggatgagcacggtgtggaacgccacgactacaactccctgggttccaggcgtggtaaccacgaggtcatgatgcgcggcaccttcgccaacatccgcctccagaaccagctggttgacatcgcaggtggctacacccgcgacttcacccaggagggtgctccacaggcgttcatctacgacgcttccgtcaactacaaggctgctggcattccgctggtcgtcttgggcggcaaggagtacggcaccggttcttcccgtgactgggcagctaagggcactaacctgctcggaattcgcgcagttatcaccgagtccttcgagcgtattcaccgctccaacctcatcggtatgggcgttgtcccactgcagttccctgcaggcgaatcccacgagtccctgggccttgacggcaccgagaccttcgacatcaccggactgaccgcactcaacgagggcgagactcctaagactgtcaaggtcaccgcaaccaaggagaacggcgacgtcgtcgagttcgacgcagttgtccgcatcgacaccccaggtgaggctgactactaccgccacggcggcatcctgcagtacgtgctgcgtcagatggctgcttcttctaagtaa

SEQ ID NO: 3 - DNA, 2676 bp, Escherichia coli
  source 1..2676: mol_type = genomic DNA
atgtcgtcaaccctacgagaagccagtaaagacacgttgcaggccaaagataaaacttaccactactacagcctgccgcttgctgccaaatcactgggcgatatcacccgtctacccaagtcactcaaagttttgctcgaaaacctgctgcgttggcaggatggtaaatcggttaccgaagaggatatccacgcgctggcgggatggctgaaaaatgcccatgctgaccgtgaaattgcctaccgcccggcaagagtgctgatgcaggactttaccggcgtacctgccgttgttgatctggcggcaatgcgcgaagcggttaaacgcctcggcggcgatactgcaaaggttaacccactctcaccggtcgacctggtcattgaccactcggtgaccgtcgatcgttttggtgatgatgaggcatttgaagaaaacgtacgcctggaaatggagcgcaaccacgaacgttatgtgttcctgaaatggggaaagcaggcgttcagtcggtttagcgtcgtgccgccaggcacaggcatttgccatcaggttaacctcgaatatctcggcaaagcagtgtggagcgaattgcaggacggtgaatggattgcttatccggatacactcgttggtactgactcgcacaccaccatgatcaacggccttggcgtgctggggtggggcgttggtggcatcgaagcagaagccgcaatgttaggccagccggtttccatgcttatcccggacgtagtgggctttaaacttaccggaaaattacgtgaaggtattaccgccacagacctggttctcactgttacccaaatgttgcgcaaacatggcgtggtggggaaattcgtcgaattttatggtgatgggctggattcactaccgctggcggatcgcgccaccattgccaatatgtcgccagaatatggtgccacctgtggcttcttcccaatcgatgctgtcaccctcgattacatgcgtttaagcgggcgcagtgaagatcaggtcgagttggtcgaaaaatatgccaaagcgcagggcatgtggcgtaatccgggcgatgaaccaatttttaccagtgtgttagaactggatatgaatgacgttgaagcgagcctggcagggccaaaacgcccacaggatcgcgttgcactgcccgatgtaccaaaagcatttgctgccagtagcgaactggaagtgaatgccacgcataaagatcgcctgccggtcgattatgttatgaacggacatcagtatcagttacctgatggcgctgtggtcattgctgcgataacgtcgtgcaccaacacctctaacccaagtgtgttgatggccgcaggcttgctggcgaaaaaagccgtaacgctgggcctcaagcggcaaccgtgggtcaaagcgtcgctggcaccgggttcgaaagtcgtttctgattatctggcaaaagcgaaactgacaccgtatctcgatgaactgggatttaaccttgtgggatacggttgtaccacctgtattggtaactctggaccgttgcccgatcctatcgaaacggcaatcaaaaaaggcgatttaaccgtcggtgcagtgctgtccggcaaccgtaactttgaaggccgtatccatccgctggttaaaaccaactggctggcctcgccgccgctggtggttgcctatgcgctggcgggaaatatgaatatcaacctggcttctgagcctatcggccatgatcgcaaaggcgagccggtatatctgaaagatatctggccatcggcacaagaaattgcccgtgcggtagaccaagtctccacagaaatgttccgcaaagagtacgcagaagtttttgaaggcacatctgagtggaaggaaattaacgtcacacgatccgatacctacggttggcaggaggattcaacctatattcgcttatcgcctttctttgatgaaatgcaggcaacaccagcacctgtggaagatattcacggtgcgcggatcctcgcaatgctgggggattcagtcaccactgaccatatctctccggcgggcagtattaagcccgacagcccggcgggtcgatatctacaaggtcggggtgttgagcggaaagactttaactcctatggttcgcggcgtggtaaccatgaagtgatgatgcgcggcaccttcgccaatattcgcatccgtaatgaaatggtgcctggcgttgaaggggggatgacgcgccatttacctgacagcgatgtcgtctctatttatgatgccgcaatgcgctataagcaggagcaaacgccgctggcggtgattgccgggaaagagtatggatcaggctccagccgtgactgggcggcaaaaggtccgcgtctgcttggtattcgtgttgtgattgccgaatcgtttgaacgaattcaccgttcgaatttaattggcatgggcatcctgccgctggaatttccacaaggtgtgacgcgtaaaacgttagagcttaccggggaagagaagattgatattggtgatctgcaaaatctacaacccggcgcgacggttccggtgacgcttacgcgcgcggatggtagccaggaagtcgtaccctgccgttgtcgtatcgacaccgcgacggagttgacctactaccagaacgacggcattttgcattatgtcattcgtaatatgttgaagtaa

SEQ ID NO: 4 - DNA, 8027 bp, synthetic construct
  misc_feature 1..8027
  source 1..8027: mol_type = other DNA
tcccttatgcgactcctgcattaggaaattaatacgactcactataggggaattgtgagcggataacaattcccctgtagaaataattttgtttaactttaataaggagatataccatgcaagcttatgaccaaacagagcgcagatagcaatgcaaaaagcggtgttaccagcgaaatttgtcattgggcaagcaatctggcaaccgatgatattccgagtgatgttctggaacgtgccaaatatctgattctggatggtattgcatgtgcatgggttggtgcacgtgttccgtggtcagaaaaatatgttcaggcaaccatgagctttgaaccgcctggtgcatgtcgtgttattggttatggccagaaactgggtccggttgcagcagcaatgaccaatagcgcatttattcaggccaccgaactggatgattaccatagcgaagcaccgctgcatagcgcaagcattgttctgcctgccgtttttgcagcaagcgaagttctggcagaacagggtaaaaccattagcggtattgatgttattctggcagccattgttggttttgaaagcggtccgcgtattggtaaagcaatttatggtagcgatctgctgaataatggttggcattgtggtgcagtttatggtgcaccggcaggcgcactggccaccggcaaactgctgggtctgacaccggatagcatggaagatgcactgggtattgcctgtacccaggcatgtggtctgatgagcgcacagtatggtggtatggttaaacgtgttcagcatggttttgcagcccgtaatggtctgctgggtggcctgctggcacatggtggttatgaagcaatgaaaggtgtgctggaacgtagctatggtggttttctgaaaatgtttaccaaaggcaatggtcgtgaacctccgtataaagaagaagaagttgttgcaggtctgggtagcttttggcatacctttaccattcgcattaaactgtatgcatgttgtggtctggttcatggtccggtggaagcaattgaaaatctgcaaggtcgttatccggaactgctgaatcgtgcaaatctgagcaatattcgtcatgttcatgttcagctgagcaccgcaagcaatagccattgcggttggattccggaagaacgtccgattagcagcattgcaggtcagatgagcgttgcatatattctggccgttcagctggttgatcagcagtgtctgctgagccagtttagcgaatttgatgataacctggaacgtccggaagtttgggatctggcacgtaaagttaccagcagccagagcgaagaatttgatcaggatggtaattgtctgagcgcaggtcgtgttcgtattgaatttaatgatggttccagcattaccgaaagcgttgaaaaaccgctgggtgttaaagaaccgatgccgaatgaacgtatcctgcataaatatcgtaccctggcaggtagcgttaccgatgaaagccgtgtgaaagaaattgaggatctggttctgggtctggatcgtctgaccgatattagtccgctgctggaactgctgaactgtccggttaaaagtccgctggtttaaactagtgaattcgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagagctcactgtgactgaaagcaagaactccttcaatgctaagagcacccttgaagttggcgacaagtcctatgactacttcgccctctctgcagtgcctggcatggagaagctgccgtactccctcaaggttctcggagagaaccttcttcgtaccgaagacggcgcaaacatcaccaacgagcacattgaggctatcgccaactgggatgcatcttccgatccaagcatcgaaatccagttcaccccagcccgtgttctcatgcaggacttcaccggtgtcccttgtgtagttgacctcgcaaccatgcgtgaggcagttgctgcactcggtggcgaccctaacgacgtcaacccactgaacccagccgagatggtcattgaccactccgtcatcgtggaggctttcggccgcccagatgcactggctaagaacgttgagatcgagtacgagcgcaacgaggagcgttaccagttcctgcgttggggttccgagtccttctccaacttccgcgttgttcctccaggaaccggtatcgtccaccaggtcaacattgagtacttggctcgcgtcgtcttcgacaacgagggccttgcatacccagatacctgcatcggtaccgactcccacaccaccatggaaaacggcctgggcatcctgggctggggcgttggtggcattgaggctgaagcagcaatgctcggccagccagtgtccatgctgatccctcgcgttgttggcttcaagttgaccggcgagatcccagtaggcgttaccgcaactgacgttgtgctgaccatcaccgaaatgctgcgcgaccacggcgtcgtccagaagttcgttgagttctacggctccggtgttaaggctgttccactggctaaccgtgcaaccatcggcaacatgtccccagagttcggctccacctgtgcgatgttcccaatcgacgaggagaccaccaagtacctgcgcctcaccggccgcccagaagagcaggttgcactggtcgaggcttacgccaaggcgcagggcatgtggctcgacgaggacaccgttgaagctgagtactccgagtacctcgagctggacctgtccaccgttgttccttccatcgctggccctaagcgcccacaggaccgcatccttctctccgaggcaaaggagcagttccgtaaggatctgccaacctacaccgacgacgctgtttccgtagacacctccatccctgcaacccgcatggttaacgaaggtggcggacagcctgaaggcggcgtcgaagctgacaactacaacgcttcctgggctggctccggcgagtccttggctactggcgcagaaggacgtccttccaagccagtcaccgttgcatccccacagggtggcgagtacaccatcgaccacggcatggttgcaattgcatccatcacctcttgcaccaacacctctaacccatccgtgatgatcggcgctggcctgatcgcacgtaaggcagcagaaaagggcctcaagtccaagccttgggttaagaccatctgtgcaccaggttcccaggttgtcgacggctactaccagcgcgcagacctctggaaggaccttgaggccatgggcttctacctctccggcttcggctgcaccacctgtattggtaactccggcccactgccagaggaaatctccgctgcgatcaacgagcacgacctgaccgcaaccgcagttttgtccggtaaccgtaacttcgagggacgtatctcccctgacgttaagatgaactacctggcatccccaatcatggtcattgcttacgcaatcgctggcaccatggacttcgacttcgagaacgaagctcttggacaggaccaggacggcaacgacgtcttcctgaaggacatctggccttccaccgaggaaatcgaagacaccatccagcaggcaatctcccgtgagctttacgaagctgactacgcagatgtcttcaagggtgacaagcagtggcaggaactcgatgttcctaccggtgacaccttcgagtgggacgagaactccacctacatccgcaaggcaccttacttcgacggcatgcctgtcgagccagtggcagtcaccgacatccagggcgcacgcgttctggctaagctcggcgactctgtcaccaccgaccacatctcccctgcttcctccattaagccaggtacccctgcagctcagtacttggatgagcacggtgtggaacgccacgactacaactccctgggttccaggcgtggtaaccacgaggtcatgatgcgcggcaccttcgccaacatccgcctccagaaccagctggttgacatcgcaggtggctacacccgcgacttcacccaggagggtgctccacaggcgttcatctacgacgcttccgtcaactacaaggctgctggcattccgctggtcgtcttgggcggcaaggagtacggcaccggttcttcccgtgactgggcagctaagggcactaacctgctcggaattcgcgcagttatcaccgagtccttcgagcgtattcaccgctccaacctcatcggtatgggcgttgtcccactgcagttccctgcaggcgaatcccacgagtccctgggccttgacggcaccgagaccttcgacatcaccggactgaccgcactcaacgagggcgagactcctaagactgtcaaggtcaccgcaaccaaggagaacggcgacgtcgtcgagttcgacgcagttgtccgcatcgacaccccaggtgaggctgactactaccgccacggcggcatcctgcagtacgtgctgcgtcagatggctgcttcttctaagtaaagatcttctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgcagcgctcttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctc

SEQ ID NO: 5 - DNA, 1987 bp, Escherichia coli
  source 1..1987: mol_type = genomic DNA
atgaatattcgtccattgcatgatcgcgtgatcgtcaagcgtaaagaagttgaaactaaatctgctggcggcatcgttctgaccggctctgcagcggctaaatccacccgcggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaatccgcgcacgacactgaacatacgaatttaaggaataaagataatggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgggtggcatgggcggcatgatgtaatga

SEQ ID NO: 6 - DNA, 7916 bp, synthetic construct
  misc_feature 1..7916
  source 1..7916: mol_type = other DNA
tcccttatgcgactcctgcattaggaaattaatacgactcactataggggaattgtgagcggataacaattcccctgtagaaataattttgtttaactttaataaggagatataccatgcaagcttatgaccaaacagagcgcagatagcaatgcaaaaagcggtgttaccagcgaaatttgtcattgggcaagcaatctggcaaccgatgatattccgagtgatgttctggaacgtgccaaatatctgattctggatggtattgcatgtgcatgggttggtgcacgtgttccgtggtcagaaaaatatgttcaggcaaccatgagctttgaaccgcctggtgcatgtcgtgttattggttatggccagaaactgggtccggttgcagcagcaatgaccaatagcgcatttattcaggccaccgaactggatgattaccatagcgaagcaccgctgcatagcgcaagcattgttctgcctgccgtttttgcagcaagcgaagttctggcagaacagggtaaaaccattagcggtattgatgttattctggcagccattgttggttttgaaagcggtccgcgtattggtaaagcaatttatggtagcgatctgctgaataatggttggcattgtggtgcagtttatggtgcaccggcaggcgcactggccaccggcaaactgctgggtctgacaccggatagcatggaagatgcactgggtattgcctgtacccaggcatgtggtctgatgagcgcacagtatggtggtatggttaaacgtgttcagcatggttttgcagcccgtaatggtctgctgggtggcctgctggcacatggtggttatgaagcaatgaaaggtgtgctggaacgtagctatggtggttttctgaaaatgtttaccaaaggcaatggtcgtgaacctccgtataaagaagaagaagttgttgcaggtctgggtagcttttggcatacctttaccattcgcattaaactgtatgcatgttgtggtctggttcatggtccggtggaagcaattgaaaatctgcaaggtcgttatccggaactgctgaatcgtgcaaatctgagcaatattcgtcatgttcatgttcagctgagcaccgcaagcaatagccattgcggttggattccggaagaacgtccgattagcagcattgcaggtcagatgagcgttgcatatattctggccgttcagctggttgatcagcagtgtctgctgagccagtttagcgaatttgatgataacctggaacgtccggaagtttgggatctggcacgtaaagttaccagcagccagagcgaagaatttgatcaggatggtaattgtctgagcgcaggtcgtgttcgtattgaatttaatgatggttccagcattaccgaaagcgttgaaaaaccgctgggtgttaaagaaccgatgccgaatgaacgtatcctgcataaatatcgtaccctggcaggtagcgttaccgatgaaagccgtgtgaaagaaattgaggatctggttctgggtctggatcgtctgaccgatattagtccgctgctggaactgctgaactgtccggttaaaagtccgctggtttaaactagtgaattcgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgatgtcgtcaaccctacgagaagccagtaaagacacgttgcaggccaaagataaaacttaccactactacagcctgccgcttgctgccaaatcactgggcgatatcacccgtctacccaagtcactcaaagttttgctcgaaaacctgctgcgttggcaggatggtaaatcggttaccgaagaggatatccacgcgctggcgggatggctgaaaaatgcccatgctgaccgtgaaattgcctaccgcccggcaagagtgctgatgcaggactttaccggcgtacctgccgttgttgatctggcggcaatgcgcgaagcggttaaacgcctcggcggcgatactgcaaaggttaacccactctcaccggtcgacctggtcattgaccactcggtgaccgtcgatcgttttggtgatgatgaggcatttgaagaaaacgtacgcctggaaatggagcgcaaccacgaacgttatgtgttcctgaaatggggaaagcaggcgttcagtcggtttagcgtcgtgccgccaggcacaggcatttgccatcaggttaacctcgaatatctcggcaaagcagtgtggagcgaattgcaggacggtgaatggattgcttatccggatacactcgttggtactgactcgcacaccaccatgatcaacggccttggcgtgctggggtggggcgttggtggcatcgaagcagaagccgcaatgttaggccagccggtttccatgcttatcccggacgtagtgggctttaaacttaccggaaaattacgtgaaggtattaccgccacagacctggttctcactgttacccaaatgttgcgcaaacatggcgtggtggggaaattcgtcgaattttatggtgatgggctggattcactaccgctggcggatcgcgccaccattgccaatatgtcgccagaatatggtgccacctgtggcttcttcccaatcgatgctgtcaccctcgattacatgcgtttaagcgggcgcagtgaagatcaggtcgagttggtcgaaaaatatgccaaagcgcagggcatgtggcgtaatccgggcgatgaaccaatttttaccagtgtgttagaactggatatgaatgacgttgaagcgagcctggcagggccaaaacgcccacaggatcgcgttgcactgcccgatgtaccaaaagcatttgctgccagtagcgaactggaagtgaatgccacgcataaagatcgcctgccggtcgattatgttatgaacggacatcagtatcagttacctgatggcgctgtggtcattgctgcgataacgtcgtgcaccaacacctctaacccaagtgtgttgatggccgcaggcttgctggcgaaaaaagccgtaacgctgggcctcaagcggcaaccgtgggtcaaagcgtcgctggcaccgggttcgaaagtcgtttctgattatctggcaaaagcgaaactgacaccgtatctcgatgaactgggatttaaccttgtgggatacggttgtaccacctgtattggtaactctggaccgttgcccgatcctatcgaaacggcaatcaaaaaaggcgatttaaccgtcggtgcagtgctgtccggcaaccgtaactttgaaggccgtatccatccgctggttaaaaccaactggctggcctcgccgccgctggtggttgcctatgcgctggcgggaaatatgaatatcaacctggcttctgagcctatcggccatgatcgcaaaggcgagccggtatatctgaaagatatctggccatcggcacaagaaattgcccgtgcggtagaccaagtctccacagaaatgttccgcaaagagtacgcagaagtttttgaaggcacatctgagtggaaggaaattaacgtcacacgatccgatacctacggttggcaggaggattcaacctatattcgcttatcgcctttctttgatgaaatgcaggcaacaccagcacctgtggaagatattcacggtgcgcggatcctcgcaatgctgggggattcagtcaccactgaccatatctctccggcgggcagtattaagcccgacagcccggcgggtcgatatctacaaggtcggggtgttgagcggaaagactttaactcctatggttcgcggcgtggtaaccatgaagtgatgatgcgcggcaccttcgccaatattcgcatccgtaatgaaatggtgcctggcgttgaaggggggatgacgcgccatttacctgacagcgatgtcgtctctatttatgatgccgcaatgcgctataagcaggagcaaacgccgctggcggtgattgccgggaaagagtatggatcaggctccagccgtgactgggcggcaaaaggtccgcgtctgcttggtattcgtgttgtgattgccgaatcgtttgaacgaattcaccgttcgaatttaattggcatgggcatcctgccgctggaatttccacaaggtgtgacgcgtaaaacgttagagcttaccggggaagagaagattgatattggtgatctgcaaaatctacaacccggcgcgacggttccggtgacgcttacgcgcgcggatggtagccaggaagtcgtaccctgccgttgtcgtatcgacaccgcgacggagttgacctactaccagaacgacggcattttgcattatgtcattcgtaatatgttgaagtaacaactatttgcttgccgggagctcggatctactagtctgcaggatctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgcagcgctcttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctc

(19) United States
(12) Patent Application Publication
(10) Pub. No.: US 2024/0132923 A1
(43) Pub. Date: Apr. 25, 2024

(54) RECOMBINANT MICROORGANISM AND A METHOD FOR ITACONIC ACID PRODUCTION

(71) Applicants: China Petrochemical Development Corporation, Taipei (Taiwan), Kaohsiung City (TW); NATIONAL CHEN KUNG UNIVERSITY, Tainan City (TW)

(72) Inventors: I-Son NG, Tainan City (TW); Jo-Shu CHANG, Tainan City (TW); Chuan-Chieh HSIANG, Tainan City (TW); Yeong-Chang CHEN, Kaohsiung City (TW); Yu-Chiao LIU, Kaohsiung City (TW); Chia-Wei TSAI, Kaohsiung City (TW)

(21) Appl. No.: 18/188,995

(22) Filed: Mar. 22, 2023

(30) Foreign Application Priority Data
Oct. 24, 2022 (TW) 111140468

(51) Int. Cl. C12P 7/46; C12N 9/88; C12N 15/52; C12N 15/70

(52) U.S. Cl. CPC C12P 7/46; C12N 9/88; C12N 15/52; C12N 15/70; C12R 2001/19; C12Y 401/01006; C12Y 402/01003

(57) ABSTRACT

Provided is a recombinant microorganism including at least two genes for producing itaconic acid and its derived monomers, and the at least two genes are located on the same expression vector. The at least two genes include one encoding cis-aconitic acid decarboxylase and the other one encoding aconitase, and the genome of the recombinant microorganism includes a gene encoding the molecular chaperone protein GroELS. Also provided is a method for producing itaconic acid by using the microorganism.

### FIELD OF THE INVENTION

The present disclosure relates to a method for itaconic acid production, and more particularly, to a method for itaconic acid production using a recombinant microorganism as a biocatalyst.

### SEQUENCE LISTING

The present application is being filed along with a Sequence Listing in electronic format. The Sequence Listing is provided as a file entitled 211224US-Sequence Listing.XML, created on Dec. 20, 2022, which is 31,534 bytes (about 30.7 KB) in size. The information in the electronic format of Sequence Listing is incorporated herein by reference in its entirety.

### BACKGROUND OF THE INVENTION

A traditional process for production and preparation of itaconic acid by a chemical method commonly uses organic solvents and thus results in waste water containing formaldehyde, which is a disadvantage to the purpose of a sustainable production.

Recently, an industrial fermentation mainly produces itaconic acid by in vivo fermentation using Aspergillus terreus, although a high concentration of itaconic acid can be obtained, the excessive byproducts of biological metabolism are disadvantageous to the industrial purification, and the long reaction time increases the production cost.

In prior arts, US 20190330665 A1 discloses a method for production of itaconic acid by using an enzyme as the biocatalyst, which produces itaconic acid or trans-aconitic acid by using a gene-modified bacterium of Pseudomonas. In addition, KR 102033217 B1 discloses a method for production of itaconic acid by using a recombinant vector containing the aconitase and the cis-aconitic acid decarboxylase as the biocatalyst in a culturing medium containing citric acid. The method disclosed in KR 102033217 B1 is a preferable one among the existing catalytic in vitro production, and itaconic acid in an amount of 41.6 g/L can be obtained within 43 hours with a yield of 0.96 g/L/h.

However, the yields of the above methods are still not ideal and cannot be put into industrial process for a large-scale production. There is still a need in the art for a method for production of itaconic acid by using a biocatalyst with enhancement of process capacity. The present disclosure provides a novel recombinant microorganism and a method for production itaconic acid by using the novel microorganism, thereby overcomes the problems of the well-known techniques.

### SUMMARY OF THE INVENTION

In order to overcome the aforementioned problems, the present disclosure provides a recombinant microorganism for production of itaconic acid and its derived monomers, comprising at least two genes located on a same expression vector, and the at least two genes comprise a gene encoding cis-aconitic acid decarboxylase and the other gene encoding aconitase, and the genome of the recombinant microorganism comprises a gene encoding a molecular chaperone protein GroELS.

In at least one embodiment of the present disclosure, wherein the expression vector includes an inducible promoter gene for controlling expression of the at least two genes.

In at least one embodiment of the present disclosure, the inducible promoter is a T7 promoter.

In at least one embodiment of the present disclosure, on the expression vector contained in the recombinant microorganism, the gene encoding cis-aconitic acid decarboxylase is a nucleotide sequence having at least 80% identity to SEQ ID NO: 1 and activity identical to SEQ ID NO: 1; the gene encoding aconitase is a nucleotide sequence having at least 80% identity to SEQ ID NO: 2 and an activity identical to SEQ ID NO: 2.

In at least one embodiment of the present disclosure, on the expression vector contained in the recombinant microorganism, the gene encoding cis-aconitic acid decarboxylase is a nucleotide sequence having at least 80% identity to SEQ ID NO: 1 and an activity identical to SEQ ID NO: 1; the gene encoding aconitase is a nucleotide sequence having at least 60% identity to SEQ ID NO: 3 and an activity identical to SEQ ID NO: 3.

In at least one embodiment of the present disclosure, in the genome of the recombinant microorganism, the gene encoding the molecular chaperone protein GroELS comprises a nucleotide sequence having at least 80% identity to SEQ ID NO: 5 and an activity identical to SEQ ID NO: 5.

In at least one embodiment of the present disclosure, the recombinant microorganism belongs to the genus of Escherichia, Klebsiella, Erwinia, Serratia, Providencia, Corynebacterium or Brevibacterium. In at least one embodiment of the present disclosure, the recombinant microorganism is a recombinant E. coli BL21 (DE3) strain.

In some embodiments of the present disclosure, the novel integrated strain provided in the present disclosure, AtCg/BD::7G, was deposited at the Deutsche Sammlung von Mikroorganismen und Zellkulturen (DSMZ) GmbH (Inhoffenstraße 7B, 38124 Braunschweig, Germany) under Accession No. DSM 34296 on Jun. 17, 2022. And, the other novel integrated strain provided in the present disclosure, AtEcN/BD::7G, was deposited at the Deutsche Sammlung von Mikroorganismen und Zellkulturen (DSMZ) GmbH (Inhoffenstraße 7B, 38124 Braunschweig, Germany) under Accession No. DSM 34297 on Jun. 17, 2022.

The present disclosure also provides a method for production of itaconic acid and its derived monomers, comprising: performing a reaction of the aforementioned recombinant in a solution containing citric acid to convert the citric acid for producing itaconic acid and its derived monomers; and isolating and obtaining the itaconic acid and its derived monomers from the solution.

In some embodiments of the method of the present disclosure, the solution is a mixed solution containing citric acid and a citrate.

In at least one embodiment of the method of the present disclosure, the reaction is performed at a pH value between 5.0 and 7.0, e.g., 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7.0. In at least one embodiment of the method of the present disclosure, the reaction is performed at a pH value between 5.0 and 6.0.

In at least one embodiment of the method of the present disclosure, the reaction is performed at a temperature between 22° C. and 37° C., e.g., 22° C., 23° C., 24° C., 25° C., 26° C., 27° C., 28° C., 29° C., 30° C., 31° C., 32° C., 33° C., 34° C., 35° C., 36° C. or 37° C. In at least one embodiment of the method of the present disclosure, the reaction is performed at a temperature between 22° C. and 30° C.

The present disclosure provides a BL21 (DE3) strain containing the plasmid pHCC-AtCg, which can achieve a catalytic activity superior to the best one among the existing in vitro catalytic production techniques. The novel integrated strains containing the plasmids of pHCC-AtCg and pHCC-AtEcN designed in the present disclosure, AtCg/BD::7G and AtEcN/BD::7G, can further enhance the overall efficiency of the production of itaconic acid by the whole cell catalytic reaction, in which the novel integrated strain of AtEcN/BD::7G can achieve the highest yield, thereby enabling a large scale production of itaconic acid through biocatalysts.

### BRIEF DESCRIPTION OF THE FIGURES

FIG. 1 shows the protein expression profiles of aconitases derived from different sources. Arrows point out the locations of the aconitase derived from E. coli (EcAcnA) and the aconitase derived from Corynebacterium glutamicum (CgAcnA) in the protein electrophoresis. Ec: E. coli MG1655; Cg: Corynebacterium glutamicum; EcN: E. coli Nissle; WC: whole cells; SP: supernatant; M: marker.

FIG. 2 shows the protein expression profiles of cis-aconitic acid decarboxylase derived from different sources. Arrows point out the locations of the cis-aconitic acid decarboxylase derived from Bacillus (BsCadA) and the cis-aconitic acid decarboxylase derived from Aspergillus terreus (AtCadA) in the protein electrophoresis. Bs: Bacillus; At: Aspergillus terreus; WC: whole cells; SP: supernatant; M: marker.

FIGS. 3A-3D show the plasmid maps of different single plasmid dual genes expression systems and the protein expression profiles thereof at different temperature. FIGS. 3A and 3B show the plasmid maps of the single plasmid dual genes expression systems (pHCC-AtCg, pHCC-AtEcN). FIG. 3C shows the expression profile of the single plasmid pHCC-AtCg at different temperatures (22° C., 30° C., 37° C.), in which arrows point out the locations of the Corynebacterium glutamicum aconitase (CgAcnA) and the Aspergillus terreus cis-aconitic acid decarboxylase (AtCadA) in the protein electrophoresis. FIG. 3D shows the expression profiles of the single plasmid pHCC-AtEcN at 30° C., in which arrows point out the locations of the E. coli Nissle aconitase (EcNAcnA) and the Aspergillus terreus cis-aconitic acid decarboxylase (AtCadA) in the protein electrophoresis. WC: whole cells; SP: supernatant; M: marker.

FIG. 4 shows the effects of the strain BL21 (DE3) containing the single plasmid (pHCC-AtCg) on the catalytic efficiencies of key enzymes in different culturing media. LB, G3Y1, G3Y2 and G6Y1 are different culturing media, and the components thereof are listed in Table 3. The rounds in the figure represent the production rates of the different strains. IA: itaconic acid.

FIG. 5 shows the plasmid map of pAH69 for preparing a competent cell pAH69/BL21(DE3).

FIG. 6 shows pHK-Km-T7-GroELS plasmid map.

FIG. 7 shows difference in catalytic activities of the dual key proteins in the BL21(DE3) strain, the novel integrated strains of AtCg/BD::7G and AtEcN/BD::7G. IA: itaconic acid. The rounds in the figure represent the production rates of the different strains.

### DETAILED DESCRIPTION

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the following examples for carrying out this disclosure without contravening its scope, for different aspects and applications. Furthermore, all ranges and values recited in the present disclosure are inclusive and combinable. Any value or point falling in the ranges recited herein, such as any integers, can be used as the lower or upper limit to derive a subrange.

Unless otherwise stated herein, the singular form “a”, “an” and “the” used in this description and the attached claims of the present disclosure should be considered to encompass a plurality of subjects.

Unless otherwise stated herein, the term “or” used in the description and the attached claims of the present disclosure comprises the meaning of “and/or”.

The present disclosure provides an expression vector comprising genes encoding production of the cis-aconitic acid decarboxylase (CadA) and the aconitase (AcnA) and a T7 promoter sequence controlling expressions of the nucleic acid of the cis-aconitic acid decarboxylase and the aconitase. The present disclosure also provides a recombinant microorganism comprising the expression vector and a method for production of itaconic acid by utilizing the recombinant microorganism, a genome of the recombinant microorganism comprises a gene encoding the molecular chaperone protein GroELS.

As used herein, the term “cis-aconitic acid decarboxylase (CadA)” refers to an enzyme which catalyzes the chemical reaction of cis-aconitic acid to form two products of itaconic acid and carbon dioxide. The EC number of CadA is EC 4.1.1.6. Cis-Aconitic acid decarboxylase, belonging to the lyase family, is a carboxylase capable of cleaving a carbon-carbon bond. Other commonly used names include cis-aconitate acid decarboxylase, CAD and cis-aconitic acid carboxy-lyase (cis-aconitate carboxy-lyase).

According to at least one embodiment of the present disclosure, the nucleotide sequence (gene) encoding the cis-aconitic acid decarboxylase is a sequence having at least 80% (e.g., at least 82%, at least 85%, at least 90%, at least 95%, at least 97%, at least 98%, at least 99%, or 100%) sequence identity to SEQ ID NO: 1 and an activity identical to SEQ ID NO: 1, for example, a protein exhibiting the activity of the cis-aconitic acid decarboxylase. In some embodiments, the nucleotide sequence encoding the cis-aconitic acid decarboxylase is the amino acid sequence of SEQ ID NO: 1.

As used herein, the term “aconitase (AcnA)” is an iron-sulfur protein, which involves in a part of the citric acid circulation and converts citrates (salts) into iso-citrates (salts). A particular form of aconitase is present in the mitochondria within a cell to perform the most conversion in the citric acid circulation, while a similar form is present in the cytoplasm to perform other reactions for production of isocitric acids. The EC number of AcnA is EC 4.2.1.3. Other commonly used names include aconitate hydratase.

As used herein, the term “molecular chaperone GroELS” is the one intensively studied among molecular chaperone systems. The chaperone GroELS forms a macromolecular cavity (Anfinsen cage) with a co-chaperone GroES under the action of ATP, thereby providing a suitable micro-environment for protein folding^{1,2}.

According to at least one embodiment of the present disclosure, the nucleotide sequence (gene) encoding the aconitase is a sequence having at least 80% (e.g., at least 82%, at least 85%, at least 90%, at least 95%, at least 97%, at least 98%, at least 99%, or 100%) sequence identity to SEQ ID NO: 2 and an activity identical to SEQ ID NO: 2, for example, a protein exhibiting the activity of the aconitase. In some embodiments, the nucleotide sequence encoding the aconitase is the amino acid sequence of SEQ ID NO: 2.

According to at least one embodiment of the present disclosure, the nucleotide sequence encoding the aconitase is a sequence having at least 60% (e.g., at least 62%, at least 65%, at least 70%, at least 75%, at least 80%, at least 85%, at least 90%, at least 95%, at least 97%, at least 98%, at least 99%, or 100%) sequence identity to SEQ ID NO: 3 and an activity identical to SEQ ID NO: 3, for example, a protein exhibiting the activity of the aconitase. In some embodiments, the nucleotide sequence encoding the aconitase is the amino acid sequence of SEQ ID NO: 3.

According to at least one embodiment of the present disclosure, the nucleotide sequence encoding the molecular chaperone protein GroELS gene is a sequence having at least 80% (e.g., at least 82%, at least 85%, at least 90%, at least 95%, at least 97%, at least 98%, at least 99%, or 100%) sequence identity to SEQ ID NO: 5 and an activity identical to SEQ ID NO: 5, for example, a protein exhibiting the activity of the molecular chaperone protein GroELS gene. In some embodiments of the present disclosure, the nucleotide sequence encoding the molecular chaperone protein GroELS is the amino acid sequence of SEQ ID NO: 5.

The expression vector provided in at least one embodiment of the present disclosures comprises a gene encoding production of the cis-aconitic acid decarboxylase (CadA) and the aconitase (AcnA) and a T7 promoter sequence controlling the nucleic acid expressions of the cis-aconitic acid decarboxylase and the aconitase. In at least one embodiment of the present disclosure, the expression vector comprises a sequence of SEQ ID NO: 4 or having at least 80% (e.g., at least 82%, at least 85%, at least 90%, at least 95%, at least 97%, at least 98%, at least 99%, or 100%) sequence identity to SEQ ID NO: 4 and an activity identical to SEQ ID NO: 4, for example, a protein exhibiting the activities of the cis-aconitic acid decarboxylase and the aconitase.

The expression vector provided in at least one embodiment of the present disclosures comprises a gene encoding production of the cis-aconitic acid decarboxylase (CadA) and the aconitase (AcnA) and a T7 promoter sequence controlling the nucleic acid expressions of the cis-aconitic acid decarboxylase and the aconitase. In at least one embodiment of the present disclosure, the expression vector comprises a sequence of SEQ ID NO: 6 or having at least 80% (e.g., at least 82%, at least 85%, at least 90%, at least 95%, at least 97%, at least 98%, at least 99%, or 100%) sequence identity to SEQ ID NO: 6 and an activity identical to SEQ ID NO: 6, for example, a protein exhibiting the activities of the cis-aconitic acid decarboxylase and the aconitase.

As used herein, the term “sequence having . . . identity to SEQ ID NO. . . . ” refers to a percentage by which the nucleotide residues of a candidate nucleic acid fragment are completely identical to those of a reference nucleic acid fragment. During the alignment, the candidate nucleic acid fragment can be firstly aligned to the reference nucleic acid fragment, and gaps can be introduced when needed to from the highest sequence identity. As for calculation of the sequence identity, the nucleotide residues in a degenerate codon would be considered as different residues, for example, it should be considered as having a different residue of U or C between the codons AAU and AAC both of which encode an aspartic acid.

It should be understood that, due to the codon degeneracy, in comparison to the nucleotide sequence used as a reference nucleic acid fragment in the present disclosure, the nucleotide sequence of a candidate nucleic acid having modifications (such as deletion, substitution or addition) in at least a part of the sequence is also within the scope of the present disclosure, as long as the resulting candidate nucleic acid fragment substantially has a biological activity identical to the nucleotides of the reference nucleic acid. For example, in the nucleotide sequence encoding the cis-aconitic acid decarboxylase of the present disclosure, various modifications can be made in the encoding region, provided that the activity of the polypeptide expressed in the encoding region remains no change. Therefore, the nucleotide sequence of the gene encoding th cis-aconitic acid decarboxylase of the present disclosure can be any nucleotide sequence having the nucleotide sequence of SEQ ID NO: 1 or having at least 80% sequence identity to SEQ ID NO: 1, as long as the protein encoded by the nucleotide sequence exhibits the activity of the cis-aconitic acid decarboxylase.

As used herein, the term “inducible promoter” refers to a promoter which must receive an external signal or inducer to regulate the expression of a particular gene.

According to at least one embodiment of the present disclosure, the expression vector of the present disclosure further comprises at least one selected from the group consisting of a sequence of a marker gene, a sequence of a reporter gene, a sequence of an antibiotic resistant gene, a sequence of a restriction enzyme cleavage site, a sequence of a polyadenylation site, a sequence of a promoter, a sequence of a terminator, and a sequence of an operator.

According to at least one embodiment of the present disclosure, the present disclosure provides a recombinant microorganism comprising at least two genes and a T7 promoter controlling the expressions of the at least two genes, the at least two genes and the T7 promoter all locate on the same expression vector, and the at least two genes include one encoding the cis-aconitic acid decarboxylase and the other one encoding the aconitase. The genome of the recombinant microorganism comprises a gene encoding the molecular chaperone protein GroELS, and the molecular chaperone protein GroELs has the functions of stabilizing and optimizing the cis-aconitic acid decarboxylase and the aconitase. In some embodiments, the recombinant microorganism provided in the present disclosure is a recombined E. coli BL21 (DE3) strain, and the recombinant E. coli BL21 (DE3) strain can express the cis-aconitic acid decarboxylase and the aconitase simultaneously. In some embodiments, the present disclosure provides a recombinant E. coli BL21 (DE3) strain for production of itaconic acid.

As used herein, the term “recombination” refers to manually combining two sequence fragments separated from each other. In general, the term “recombination” refers to a nucleic acid, a protein or a microorganism which contains or is encoded by genetic materials from different sources, such as from organisms of two or more different lineages or species.

As used herein, the term “in vitro whole-cell biotransformation” refers to a method for performing a chemical conversion by utilizing a whole biological organism as a catalyst, i.e., the microorganisms are cultured to a certain amount of cells, then a substrate is added to perform catalysis for conversion of a product. For example, the recombinant microorganism is allowed to express cis-aconitic acid decarboxylase and aconitase simultaneously and the amount of bacteria is enhanced through a high concentration fermentation process, thereby the expression amounts of the cis-aconitic acid decarboxylase and aconitase are increased, and thus the conversion efficiency of the catalytic production of itaconic acid is improved.

As used herein, the term “microorganism” pertains to a microscopic organism and includes a bacterium, an archaebacteria, a virus or a fungus. As used herein, the reference to “microorganism” should be understood to encompass “bacteria”.

The microorganism hosts suitable for use as the expression vectors of the present disclosure include, but not limited to, the microorganisms belonging to the genus of Escherichia, Klebsiella, Erwinia, Serratia, Providencia, Corynebacterium or Brevibacterium. In some embodiments, the microorganism host used in the present disclosure expresses cis-aconitic acid decarboxylase and aconitase in vivo.

The features and effects will be further illustrated in below by particular embodiments which are not intended to limit the scope of the present disclosure.

### EXAMPLES

Strains Culturing

Strain activating: Stains stored at −80° C. were seeded into a LB plating medium containing kanamycin (Km) for activation and were cultured at 37° C. for 12 hrs.

Pre-activation culturing: the strains in the aforementioned activating step were seeded into 4 mL LB liquid medium for continuous culturing, 50 mg/L of Km was added, and culturing was performed under a temperature of 37° C. for 8 hrs.

Pre-culturing: the strains were transferred to 20 mL LB liquid medium at a seeding quantity of 1%, 50 mg/L of Km was added, and culturing was performed under a temperature of 37° C. for 12 hrs.

Induction culturing: an M9 medium was added in a total volume of 50 mL at a seeding quantity of 2%, and 50 mg/L of Km was added. Culturing was performed under a temperature of 37° C. until the biomass reached OD600 of 0.6-0.8, at which time 0.1 mM isopropyl-β-D-thiogalactoside inducer (IPTG) was added for induction, then culturing was performed at a temperature of 22-37° C. for 12-16 hrs.

Whole-Cell Conversion Activity Analysis

The bacterial cells produced were washed twice with deionized water, the desired bacterial biomass was collected in a centrifuge tube and centrifuged at a rotation rate of 12,000 rpm, and the supernatant in the centrifuge tube was removed. To each centrifuge tube, 1 mL of 1 M citric acid/citrate mixed solution with pH 5.5 was added to disperse the bacterial cells, the mixture was charged into a 2 mL centrifuge tube, and the tube clamped with an explosion-proof clip was placed in an incubator at 37° C. for reacting. After reacting for 8 hrs, the centrifuge tube was collected and centrifuged at a rotation rate of 12,000 rpm, and the supernatant was drawn to prepare an HPLC sample for a quantitative analysis.

The 1 M citric acid/citrate mixed solution with pH 5.5 was formulated as following:
1 M Citric acid solution: 9.6 g anhydrous citric acid (SHOWA Chemical Industry Co, Ltd, Japan) was added into water to a volume of 50 mL.
1 M Citrate solution: 14.7 g sodium citrate dihydrate (PANREAC, Europe) was added into water to a volume of 50 mL.
1 M Citric acid aqueous solution was added into 1 M citrate solution aqueous solution, and the mixed solution was adjusted to pH 5.5.

Quantitative analysis of itaconic acid and aconitic acid production by HPLC

In order to quantifying yields of itaconic acid (itaconic acid, IA) and aconitic acid (aconitic acid, CAA), after reaction, the supernatant was diluted for 50× and filtered, and then was separated by using a high performance liquid chromatography (HPLC, Hitachi, Japan) on a Coregel 87H3 column eluting with a mobile phase of 0.008 N H_2SO_4 (0.008 N H₂SO₄: 440 μL of pure sulfuric acid (ECHO CHEMICAL CO., LTD., Taiwan) in 2 L deionized water) at a flow rate of 0.4 mL/min and at a column temperature of 70° C. The retention times of aconitic acid (CAA), citric caid (CA), and itaconic acid (IA) were 10.8 min, 12.1 min, and 18.8 min, respectively, detected at an ultraviolet wavelength of 210 nm. The results were brought into a calibration curve for calculation to obtain the yields and production rates.

### Example 1: Screening of Cis-Aconitic Acid Decarboxylase CadA and Aconitase AcnA Genes

In order to construct an inducible promoter, T7 promoter, to express target genes such as E. coli MG1655 aconitase (EcAcnA), Corynebacterium glutamicum aconitase (CgAcnA), E. coli Nissle aconitase (EcNAcnA), Bacillus cis-aconitic acid decarboxylase (BsCadA) and Aspergillus terreus cis-aconitic acid decarboxylase (AtCadA), cleavage site primers carrying HindIII and PstI were firstly designed. A polymerase chain reaction (PCR) was performed using a genome as the template to obtain an amplified relative gene fragment, then the target gene fragment was cloned into the pHCC-duet vector by an enzyme digestion reaction, and the vector was transformed into a constructive strain DH5a. The sequence of the gene fragment encoding the Aspergillus terreus cis-aconitic acid decarboxylase (AtCadA) was the sequence as shown in SEQ ID NO: 1, the sequence of the gene fragment encoding the Corynebacterium glutamicum aconitase (CgAcnA) was the sequence as shown in SEQ ID NO: 2, and the sequence of the gene fragment encoding the E. coli Nissle aconitase (EcNAcnA) was the sequence as shown in SEQ ID NO: 3 (see Table 4).

The completely constructed plasmids were sent into E. coli BL21 (DE3)-competent cells and were screened in a culturing medium containing kanamycin. After culturing, a protein analysis for relative protein expression was performed on a sodium dodecyl sulfate polyacrylamide gel electrophoresis (SDS-PAGE) and an activity test was performed.

Here, steps for SDS-PAGE protein analysis were further described. 30 μL Protein sample to be tested for enzyme activity was taken and mixed with 10 μL protein dye, heated at 100° C. for 10 minutes, and 15 μL sample solution was added into the film sample sink for electrophoresis.

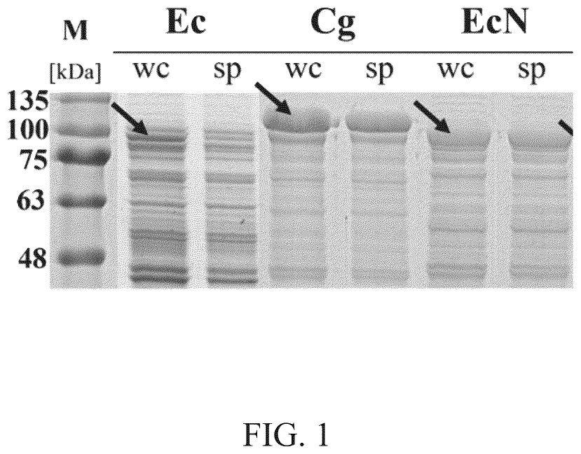

As shown in FIG. 1, it can be seen from the results of SDS-PAGE protein electrophoresis, in comparison to the protein expression quantity of the aconitase derived from E. coli M G1655 (EcAcnA), the protein expression quantity of the aconitase derived from Corynebacterium glutamicum (CgAcnA) is much higher. Additionally, the expression quantity of the aconitase derived from E. coli Nissle (EcNAcnA) is close to that of the aconitase derived from Corynebacterium glutamicum (CgAcnA), and the protein expression of aconitase is concentrated in the cell regardless of whether th aconitase is derived from E. coli or Corynebacterium glutamicum.

Table 1 shows yields of cis-aconitic acid (CAA) from citric acid (CA) by the action of the E. coli aconitase (EcAcnA) and the Corynebacterium glutamicum aconitase (CgAcnA) under different pH values for two hours. It can be seen that CgAcnA gives a significantly increased yield at a pH of 5.5-7.0, and thus exhibits an enzyme catalytic capacity superior to the EcAcnA.

TABLE 1
Yields of CAA produced from CA by EcAcnA
and CgAcnA at different pH values

| | | | | | |
|---|---|---|---|---|---|
| CAA (g/L) | pH | 5.0 | 5.5 | 6.0 | 7.0 |
| EcAcnA | OD 5 | 0.008 | 0.29 | 0.65 | 0.51 |
| | OD 10 | 0.13 | 0.39 | 0.93 | 0.58 |
| CgAcnA | OD 5 | 0.49 | 1.34 | 3.65 | 3.53 |
| | OD 10 | 0.56 | 4.06 | 4.46 | 4.72 |

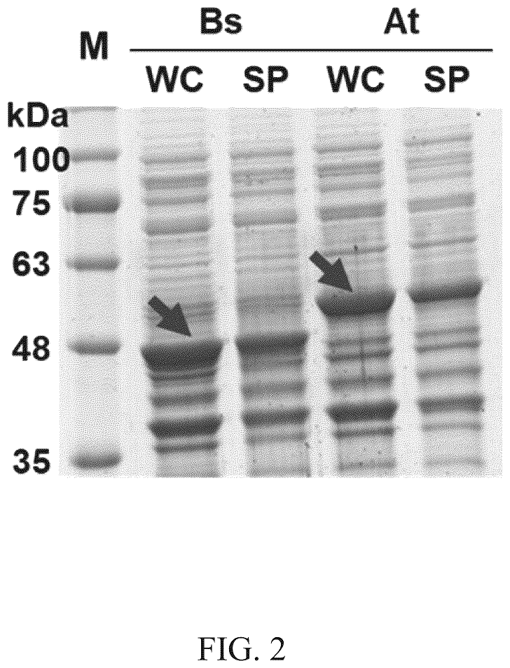

As shown in FIG. 2, it can be seen from the results of SDS-PAGE protein electrophoresis, both the cis-aconitic acid decarboxylase derived from Bacillus (BsCadA) and the cis-aconitic acid decarboxylase derived from Aspergillus terreus (AtCadA) have significant protein expression quantity, and the protein expression of the cis-aconitic acid decarboxylase is mainly present in the supernatant regardless of whether the cis-aconitic acid decarboxylase is derived from Bacillus or Aspergillus terreus.

Table 2 shows yields of itaconic acid (IA) from cis-aconitic acid (CAA) by the action of the Bacillus cis-aconitic acid decarboxylase (BsCadA) and the Aspergillus terreus cis-aconitic acid decarboxylase (AtCadA) under different pH values for two hours. It can be seen that AtCadA exhibits an advantageous enzyme catalytic capacity at a pH ranging from 5.6 to 6.0.

TABLE 2
Yields of IA produced from CAA by BsCadA
and AtCadA at different pH values

| IA (g/L) | pH | 4.8 | 5.3 | 5.6 | 5.8 | 5.9 |
|---|---|---|---|---|---|---|
| BsCadA | OD 5 | 0.001 | 0.001 | 0.002 | 0.001 | 0.002 |
| | OD 10 | 0.000 | 0.000 | 0.000 | 0.001 | 0.002 |
| AtCadA | OD 5 | 0.15 | 0.66 | 2.19 | 2.75 | 2.69 |
| | OD 10 | 0.35 | 0.61 | 2.49 | 3.22 | 3.19 |

### Example 2: Construction of a Single Plasmid Dual Genes Expression System

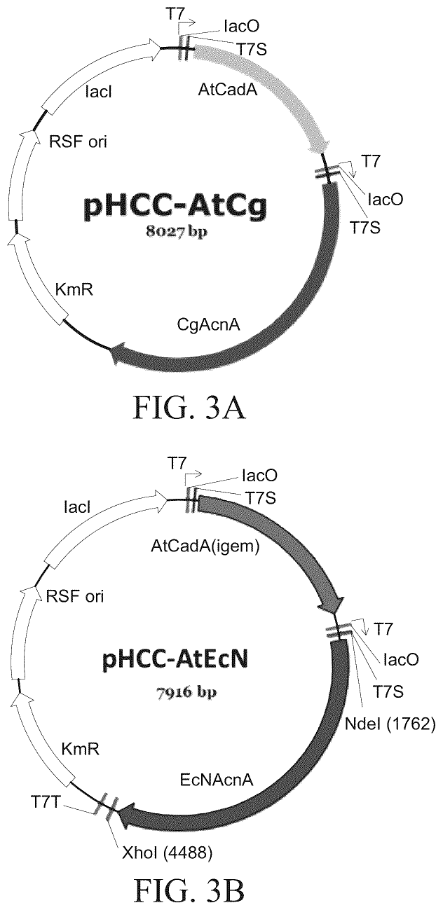

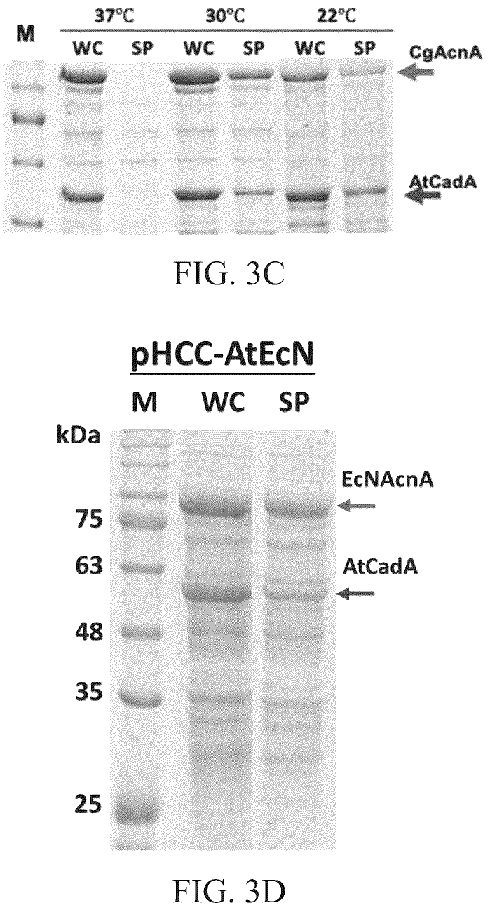

According to the results of Example 1, the aconitase derived from Corynebacterium glutamicum (CgAcnA) or the aconitase derived from E. coli Nissle (EcNAcnA) and the cis-aconitic acid decarboxylase derived from Aspergillus terreus (AtCadA) were chosen to construct a single plasmid dual genes expression system, by using a pHCC-duet as the vector and inserting the CgAcnA gene or the EcNAcnA gene into the vector pHCC-AtCadA using a prolonged overlay extension polymerase chain reaction (POE-PCR). Finally, an expression plasmid pHCC-AtCg or pHCC-AtEcN containing the dual genes described above was obtained, which have plasmid sizes of 8027 bps and 7916 bps, respectively, and contain single restriction sites: HindIII, SpeI, PstI, BglII, SalI, XhoI, SacI, NdeI, NotI and the like. The plasmids pHCC-AtCg and pHCC-AtEcN have sequences as SEQ ID NO: 4 and SEQ ID NO: 6, respectively (see Table 4) and plasmid maps set forth in FIGS. 3A, 3B.

FIG. 3C shows the expression profiles of the expression plasmid pHCC-AtCg containing AtCadA and CgAcnA dual genes at different temperatures (22° C., 30° C., 37° C.). It can be seen that pHCC-AtCg can express the Aspergillus terreus cis-aconitic acid decarboxylase (AtCadA) and the Corynebacterium glutamicum aconitase (CgAcnA) in the BL21(DE3) strain at a temperature in the range of from 22° C. to 30° C.

FIG. 3D shows the expression profiles of the expression plasmid pHCC-AtEcN containing the AtCadA and the EcNAcnA dual genes at 30° C. It can be seen that the pHCC-AtEcN can express the Aspergillus terreus cis-aconitic acid decarboxylase (AtCadA) and the E. coli Nissle aconitase (EcNAcnA) in the BL21(DE3) strain.

### Example 3: Effects of Different Culturing Media on the Catalytic Effects of the Single Plasmid Dual Genes Expression System

Culturing media of different components (Table 3) were tested for effects on the key enzyme catalytic effects of the single plasmid dual genes expression system, by transforming the plasmid pHCC-AtCg into E. coli BL21(DE3) strains, performing bacterial cells culturing the transformed BL21(DE3) strains containing the plasmid pHCC-AtCg, thereafter, performing the in vitro whole-cell biotransformation of 1 M citric acid with pH 5.5. After reacting for 8 hrs, collecting the supernatant for HPLC to quantitatively analyze the yields and production rates of the produced itaconic acid.

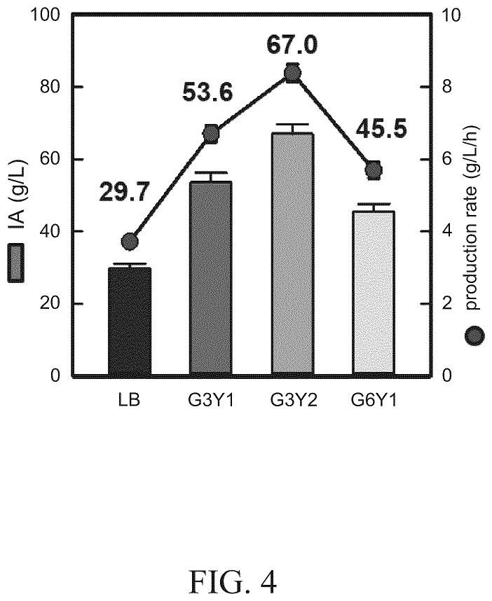

As shown by results in FIG. 4, the culture of BL21(DE3) strains containing the plasmid pHCC-AtCg in an LB, G3Y1, G3Y2, G6Y1 medium, respectively, and the in vitro whole-cell biotransformation of 1 M citric acid, pH 5.5 for 8 hrs give itaconic acid at a yield of 29.7, 53.6, 67.0, 45.5 g/L, respectively, and a production rate of 3.7125, 6.7, 8.375, 5.6875 g/L/h, respectively, suggesting that the strains cultured in the G3Y2 medium has the highest yield (67.0 g/L) and production rate (8.375 g/L/h) for itaconic acid.

TABLE 3
Compositions of different main culture media

| | LB(BD, | | | |
|---|---|---|---|---|
| (g/L)\Medium | USA) | G3Y1 | G3Y2 | G6Y2 |
| Tryptone (MDBio, Inc., Taiwan) | 10 | | | |
| Glucose | | | | 10 |
| Glycerol | | 10 | 10 | |
| Yeast Extract (BD, USA) | 5 | 1 | 2 | 2 |
| NaCl (SHOWA CHEMICAL | 10 | 0.5 | 0.5 | 0.5 |
| INDUSTRY Co, Ltd, Japan) | | | | |
| Na₂HPO₄•12H₂O (SHOWA | | 17.1 | 17.1 | 17.1 |
| CHEMICAL INDUSTRY | | | | |
| Co, Ltd, Japan) | | | | |
| K₂HPO₄ (SHOWA CHEMICAL | | 3 | 3 | 3 |
| INDUSTRY Co, Ltd, Japan) | | | | |
| NH₄Cl | | 1 | 1 | 1 |
| MgSO₄ (J T Baker, USA) | | 0.24 | 0.24 | 0.24 |
| CaCl₂ (J T Baker, USA) | | 0.01 | 0.01 | 0.01 |

### Example 4: Construction of the Novel Integrated Strains AtCg/BD::7G and AtEcN/BD::7G for Production of Itaconic Acid

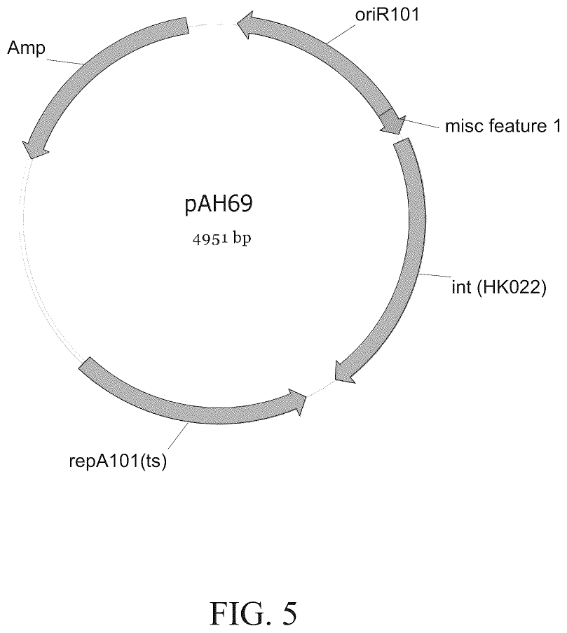

In order to constructing the novel integrated strains AtCg/BD::7G and AtEcN/BD::7G for production of itaconic acid, a gene containing a molecular chaperone protein GroELS in its genome is required to be constructed first. A pAH69 plasmid was transformed into an E. coli. BL21(DE) strain to produce a pAH69BL21(DE3)-competent cell for the strain. The pAH69 plasmid is a helper plasmid for integration at the site of HK022. The pAH69 plasmid is necessary for the genome integration of pIT5_KH plasmid³, and the plasmid map thereof is shown in FIG. 5.

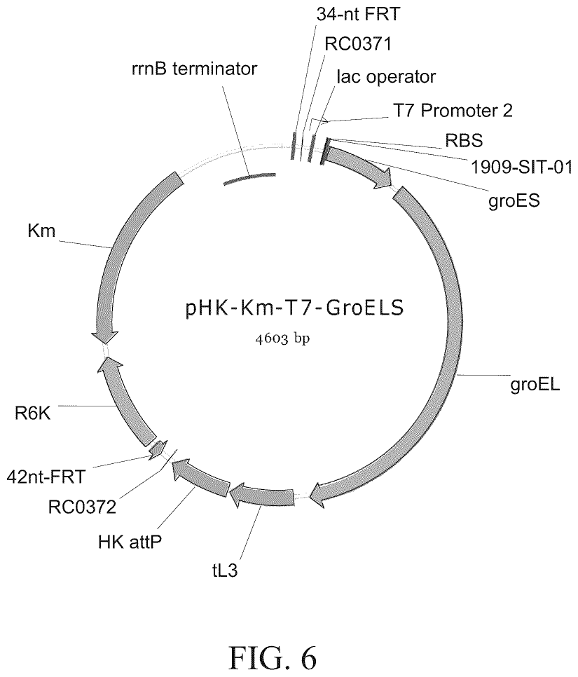

Thereafter, the pHK-Km-T7-GroELS plasmid (the plasmid map thereof was shown in FIG. 6) was transformed into the pAH69/BL21(DE3)-competent cells by a heat shock transformation process, after transformation completed, the cells were plated in solid plate culture medium containing antibiotics and cultured at 37° C. for 24 hrs. A colony on the plate culture medium was picked for PCR verification, to afford the BD::7G integrated strains.

The pHCC-AtCg and the pHCC-AtEcN plasmids constructed in Example 2 were transformed into BD::7G integrated strains by heat shock transformation, respectively, then the completely transformed cells were plated in a solid plating medium containing antibiotics and cultured at 37° C. for 24 hrs to afford novel integrated strains AtCg/BD::7G and AtEcN/BD::7G containing the single plasmid dual genes expression system.

### Example 5: The Catalytic Effects of the Novel Integrated Strains AtCg/BD::7G and AtEcN/BD::7G Containing the Single Plasmid Dual Genes Expression System

By the method of Example 4, novel integrated strains AtCg/BD::7G and AtEcN/BD::7G containing the single plasmid dual genes expression system were constructed. The novel integrated strains AtCg/BD::7G, AtEcN/BD::7G as well as the BL21(DE3) strain containing the plasmid pHCC-AtCg alone (free of molecular chaperone protein GroELS gene) was subjected to bacterial cells culturing in a G3Y2 medium, respectively. Then in vitro whole-cell biotransformations of 1 M citric acid, pH 5.5 were performed, respectively. After reacting for 8 hrs, the supernatant was collected for HPLC to quantitatively analyze the yields and production rates of itaconic acid.

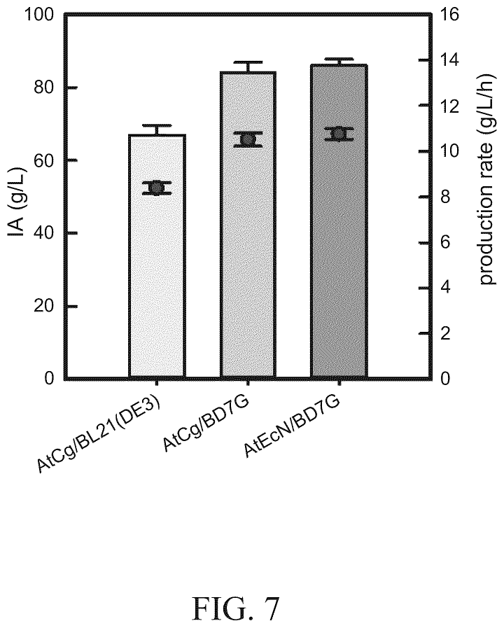

As shown by the results in FIG. 7, when the novel integrated strains AtCg/BD::7G and AtEcN/BD::7G designed in the present disclosure were utilized to perform the in vitro whole-cell biotransformations of 1 M citric acid, pH 5.5 for 8 hrs, the catalytic capacities of the AtCg/BD::7G and the AtEcN/BD::7G give final yields of itaconic acid of 84.0 and 86.0 g/L, and production rates of 10.5 and 10.75 g/L/h, respectively, suggesting a significantly improved catalytic activity (with the yield of itaconic acid being 67.0 g/L and the production rate being 8.375 g/L/h) compared to the BL21(DE3) containing the plasmid pHCC-AtCg.

The BL21(DE3) strains containing the plasmid pHCC-AtCg cultured in a G3Y2 medium of the present disclosure has a catalytic activity significantly superior to the best one (e.g., as described in KR 102033217 B1, the yield of itaconic acid over 43 hrs of 41.6 g/L and the production rate of 0.96 g/L/h, or the yield in Halomonas bluephagenesis of 63.3 g/L) of the existing in vitro biotransformation techniques. The applicant surprisingly found that the novel integrated strains AtCg/BD::7G and AtEcN/BD::7G containing the single plasmid dual genes expression system designed in the present disclosure can further improve the overall efficiency of the production of itaconic acid by the in vitro whole-cell biotransformation, with a high catalytic activity (the highest yield of itaconic acid being 86.0 g/L and the highest production rate being 10.75 g/L/h).

The related nucleotide sequences of AtCadA, CgAcnA, EcNAcnA, plasmids pHCC-AtCg and pHCC-AtEcN and gene sequence of GroELS are listed in detail in Table 4, below.

TABLE 4

| Gene, Plasmid | Nucleotide sequence |
|---|---|
| AtCadA | atgaccaaacagagcgcagatagcaatgcaaaaagcggtgttaccagcgaaatttgtcattgggcaagcaatctg |
| (SEQ ID NO: | gcaaccgatgatattccgagtgatgttctggaacgtgccaaatatctgattctggatggtattgcatgtgcatgggtt |
| 1) | ggtgcacgtgttccgtggtcagaaaaatatgttcaggcaaccatgagctttgaaccgcctggtgcatgtcgtgttatt |
| | ggttatggccagaaactgggtccggttgcagcagcaatgaccaatagcgcatttattcaggccaccgaactggat |
| | gattaccatagcgaagcaccgctgcatagcgcaagcattgttctgcctgccgtttttgcagcaagcgaagttctggc |
| | agaacagggtaaaaccattagcggtattgatgttattctggcagccattgttggttttgaaagcggtccgcgtattgg |
| | taaagcaatttatggtagcgatctgctgaataatggttggcattgtggtgcagtttatggtgcaccggcaggcgcact |
| | ggccaccggcaaactgctgggtctgacaccggatagcatggaagatgcactgggtattgcctgtacccaggcat |
| | gtggtctgatgagcgcacagtatggtggtatggttaaacgtgttcagcatggttttgcagcccgtaatggtctgctgg |
| | gtggcctgctggcacatggtggttatgaagcaatgaaaggtgtgctggaacgtagctatggtggttttctgaaaatg |
| | tttaccaaaggcaatggtcgtgaacctccgtataaagaagaagaagttgttgcaggtctgggtagcttttggcatac |
| | ctttaccattcgcattaaactgtatgcatgttgtggtctggttcatggtccggtggaagcaattgaaaatctgcaaggt |
| | cgttatccggaactgctgaatcgtgcaaatctgagcaatattcgtcatgttcatgttcagctgagcaccgcaagcaat |
| | agccattgcggttggattccggaagaacgtccgattagcagcattgcaggtcagatgagcgttgcatatattctggc |
| | cgttcagctggttgatcagcagtgtctgctgagccagtttagcgaatttgatgataacctggaacgtccggaagtttg |
| | ggatctggcacgtaaagttaccagcagccagagcgaagaatttgatcaggatggtaattgtctgagcgcaggtcg |
| | tgttcgtattgaatttaatgatggttccagcattaccgaaagcgttgaaaaaccgctgggtgttaaagaaccgatgcc |
| | gaatgaacgtatcctgcataaatatcgtaccctggcaggtagcgttaccgatgaaagccgtgtgaaagaaattgag |
| | gatctggttctgggtctggatcgtctgaccgatattagtccgctgctggaactgctgaactgtccggttaaaagtccg |
| | ctggtttaa |
| CgAcnA | gagctcactgtgactgaaagcaagaactccttcaatgctaagagcacccttgaagttggcgacaagtcctatgact |
| (SEQ ID NO: | acttcgccctctctgcagtgcctggcatggagaagctgccgtactccctcaaggttctcggagagaaccttcttcgt |
| 2) | accgaagacggcgcaaacatcaccaacgagcacattgaggctatcgccaactgggatgcatcttccgatccaag |
| | catcgaaatccagttcaccccagcccgtgttctcatgcaggacttcaccggtgtcccttgtgtagttgacctcgcaa |
| | ccatgcgtgaggcagttgctgcactcggtggcgaccctaacgacgtcaacccactgaacccagccgagatggtc |
| | attgaccactccgtcatcgtggaggctttcggccgcccagatgcactggctaagaacgttgagatcgagtacgag |
| | cgcaacgaggagcgttaccagttcctgcgttggggttccgagtccttctccaacttccgcgttgttcctccaggaac |
| | cggtatcgtccaccaggtcaacattgagtacttggctcgcgtcgtcttcgacaacgagggccttgcatacccagat |
| | acctgcatcggtaccgactcccacaccaccatggaaaacggcctgggcatcctgggctggggcgttggtggcat |
| | tgaggctgaagcagcaatgctcggccagccagtgtccatgctgatccctcgcgttgttggcttcaagttgaccggc |
| | gagatcccagtaggcgttaccgcaactgacgttgtgctgaccatcaccgaaatgctgcgcgaccacggcgtcgtc |
| | cagaagttcgttgagttctacggctccggtgttaaggctgttccactggctaaccgtgcaaccatcggcaacatgtc |
| | cccagagttcggctccacctgtgcgatgttcccaatcgacgaggagaccaccaagtacctgcgcctcaccggcc |
| | gcccagaagagcaggttgcactggtcgaggcttacgccaaggcgcagggcatgtggctcgacgaggacaccg |
| | ttgaagctgagtactccgagtacctcgagctggacctgtccaccgttgttccttccatcgctggccctaagcgccca |
| | caggaccgcatccttctctccgaggcaaaggagcagttccgtaaggatctgccaacctacaccgacgacgctgtt |
| | tccgtagacacctccatccctgcaacccgcatggttaacgaaggtggcggacagcctgaaggcggcgtcgaag |
| | ctgacaactacaacgcttcctgggctggctccggcgagtccttggctactggcgcagaaggacgtccttccaagc |
| | cagtcaccgttgcatccccacagggtggcgagtacaccatcgaccacggcatggttgcaattgcatccatcacct |
| | cttgcaccaacacctctaacccatccgtgatgatcggcgctggcctgatcgcacgtaaggcagcagaaaagggc |
| | ctcaagtccaagccttgggttaagaccatctgtgcaccaggttcccaggttgtcgacggctactaccagcgcgca |
| | gacctctggaaggaccttgaggccatgggcttctacctctccggcttcggctgcaccacctgtattggtaactccg |
| | gcccactgccagaggaaatctccgctgcgatcaacgagcacgacctgaccgcaaccgcagttttgtccggtaac |
| | cgtaacttcgagggacgtatctcccctgacgttaagatgaactacctggcatccccaatcatggtcattgcttacgc |
| | aatcgctggcaccatggacttcgacttcgagaacgaagctcttggacaggaccaggacggcaacgacgtcttcct |
| | gaaggacatctggccttccaccgaggaaatcgaagacaccatccagcaggcaatctcccgtgagctttacgaag |
| | ctgactacgcagatgtcttcaagggtgacaagcagtggcaggaactcgatgttcctaccggtgacaccttcgagtg |
| | ggacgagaactccacctacatccgcaaggcaccttacttcgacggcatgcctgtcgagccagtggcagtcaccg |
| | acatccagggcgcacgcgttctggctaagctcggcgactctgtcaccaccgaccacatctcccctgcttcctccat |
| | taagccaggtacccctgcagctcagtacttggatgagcacggtgtggaacgccacgactacaactccctgggttc |
| | caggcgtggtaaccacgaggtcatgatgcgcggcaccttcgccaacatccgcctccagaaccagctggttgaca |
| | tcgcaggtggctacacccgcgacttcacccaggagggtgctccacaggcgttcatctacgacgcttccgtcaact |
| | acaaggctgctggcattccgctggtcgtcttgggcggcaaggagtacggcaccggttcttcccgtgactgggcag |
| | ctaagggcactaacctgctcggaattcgcgcagttatcaccgagtccttcgagcgtattcaccgctccaacctcatc |
| | ggtatgggcgttgtcccactgcagttccctgcaggcgaatcccacgagtccctgggccttgacggcaccgagac |
| | cttcgacatcaccggactgaccgcactcaacgagggcgagactcctaagactgtcaaggtcaccgcaaccaag |
| | gagaacggcgacgtcgtcgagttcgacgcagttgtccgcatcgacaccccaggtgaggctgactactaccgcca |
| | cggcggcatcctgcagtacgtgctgcgtcagatggctgcttcttctaagtaa |
| EcNAcnA | atgtcgtcaaccctacgagaagccagtaaagacacgttgcaggccaaagataaaacttaccactactacagcctg |
| (SEQ ID NO: | ccgcttgctgccaaatcactgggcgatatcacccgtctacccaagtcactcaaagttttgctcgaaaacctgctgcg |
| 3) | ttggcaggatggtaaatcggttaccgaagaggatatccacgcgctggcgggatggctgaaaaatgcccatgctg |
| | accgtgaaattgcctaccgcccggcaagagtgctgatgcaggactttaccggcgtacctgccgttgttgatctggc |
| | ggcaatgcgcgaagcggttaaacgcctcggcggcgatactgcaaaggttaacccactctcaccggtcgacctgg |
| | tcattgaccactcggtgaccgtcgatcgttttggtgatgatgaggcatttgaagaaaacgtacgcctggaaatggag |
| | cgcaaccacgaacgttatgtgttcctgaaatggggaaagcaggcgttcagtcggtttagcgtcgtgccgccaggc |
| | acaggcatttgccatcaggttaacctcgaatatctcggcaaagcagtgtggagcgaattgcaggacggtgaatgg |
| | attgcttatccggatacactcgttggtactgactcgcacaccaccatgatcaacggccttggcgtgctggggtggg |
| | gcgttggtggcatcgaagcagaagccgcaatgttaggccagccggtttccatgcttatcccggacgtagtgggctt |
| | taaacttaccggaaaattacgtgaaggtattaccgccacagacctggttctcactgttacccaaatgttgcgcaaac |
| | atggcgtggtggggaaattcgtcgaattttatggtgatgggctggattcactaccgctggcggatcgcgccaccatt |
| | gccaatatgtcgccagaatatggtgccacctgtggcttcttcccaatcgatgctgtcaccctcgattacatgcgtttaa |
| | gcgggcgcagtgaagatcaggtcgagttggtcgaaaaatatgccaaagcgcagggcatgtggcgtaatccggg |
| | cgatgaaccaatttttaccagtgtgttagaactggatatgaatgacgttgaagcgagcctggcagggccaaaacgc |
| | ccacaggatcgcgttgcactgcccgatgtaccaaaagcatttgctgccagtagcgaactggaagtgaatgccacg |
| | cataaagatcgcctgccggtcgattatgttatgaacggacatcagtatcagttacctgatggcgctgtggtcattgct |
| | gcgataacgtcgtgcaccaacacctctaacccaagtgtgttgatggccgcaggcttgctggcgaaaaaagccgta |
| | acgctgggcctcaagcggcaaccgtgggtcaaagcgtcgctggcaccgggttcgaaagtcgtttctgattatctg |
| | gcaaaagcgaaactgacaccgtatctcgatgaactgggatttaaccttgtgggatacggttgtaccacctgtattgg |
| | taactctggaccgttgcccgatcctatcgaaacggcaatcaaaaaaggcgatttaaccgtcggtgcagtgctgtcc |
| | ggcaaccgtaactttgaaggccgtatccatccgctggttaaaaccaactggctggcctcgccgccgctggtggttg |
| | cctatgcgctggcgggaaatatgaatatcaacctggcttctgagcctatcggccatgatcgcaaaggcgagccgg |
| | tatatctgaaagatatctggccatcggcacaagaaattgcccgtgcggtagaccaagtctccacagaaatgttccg |
| | caaagagtacgcagaagtttttgaaggcacatctgagtggaaggaaattaacgtcacacgatccgatacctacggt |
| | tggcaggaggattcaacctatattcgcttatcgcctttctttgatgaaatgcaggcaacaccagcacctgtggaagat |
| | attcacggtgcgcggatcctcgcaatgctgggggattcagtcaccactgaccatatctctccggcgggcagtatta |
| | agcccgacagcccggcgggtcgatatctacaaggtcggggtgttgagcggaaagactttaactcctatggttcgc |
| | ggcgtggtaaccatgaagtgatgatgcgcggcaccttcgccaatattcgcatccgtaatgaaatggtgcctggcgt |
| | tgaaggggggatgacgcgccatttacctgacagcgatgtcgtctctatttatgatgccgcaatgcgctataagcag |
| | gagcaaacgccgctggcggtgattgccgggaaagagtatggatcaggctccagccgtgactgggcggcaaaa |
| | ggtccgcgtctgcttggtattcgtgttgtgattgccgaatcgtttgaacgaattcaccgttcgaatttaattggcatggg |
| | catcctgccgctggaatttccacaaggtgtgacgcgtaaaacgttagagcttaccggggaagagaagattgatatt |
| | ggtgatctgcaaaatctacaacccggcgcgacggttccggtgacgcttacgcgcgcggatggtagccaggaagt |
| | cgtaccctgccgttgtcgtatcgacaccgcgacggagttgacctactaccagaacgacggcattttgcattatgtca |
| | ttcgtaatatgttgaagtaa |
| pHCC-AtCg | tcccttatgcgactcctgcattaggaaattaatacgactcactataggggaattgtgagcggataacaattcccctgt |
| (SEQ ID NO: | agaaataattttgtttaactttaataaggagatataccatgcaagcttatgaccaaacagagcgcagatagcaatgca |
| 4) | aaaagcggtgttaccagcgaaatttgtcattgggcaagcaatctggcaaccgatgatattccgagtgatgttctgga |
| | acgtgccaaatatctgattctggatggtattgcatgtgcatgggttggtgcacgtgttccgtggtcagaaaaatatgtt |
| | caggcaaccatgagctttgaaccgcctggtgcatgtcgtgttattggttatggccagaaactgggtccggttgcag |
| | cagcaatgaccaatagcgcatttattcaggccaccgaactggatgattaccatagcgaagcaccgctgcatagcg |
| | caagcattgttctgcctgccgtttttgcagcaagcgaagttctggcagaacagggtaaaaccattagcggtattgat |
| | gttattctggcagccattgttggttttgaaagcggtccgcgtattggtaaagcaatttatggtagcgatctgctgaata |
| | atggttggcattgtggtgcagtttatggtgcaccggcaggcgcactggccaccggcaaactgctgggtctgacac |
| | cggatagcatggaagatgcactgggtattgcctgtacccaggcatgtggtctgatgagcgcacagtatggtggtat |
| | ggttaaacgtgttcagcatggttttgcagcccgtaatggtctgctgggtggcctgctggcacatggtggttatgaag |
| | caatgaaaggtgtgctggaacgtagctatggtggttttctgaaaatgtttaccaaaggcaatggtcgtgaacctccg |
| | tataaagaagaagaagttgttgcaggtctgggtagcttttggcatacctttaccattcgcattaaactgtatgcatgttg |
| | tggtctggttcatggtccggtggaagcaattgaaaatctgcaaggtcgttatccggaactgctgaatcgtgcaaatc |
| | tgagcaatattcgtcatgttcatgttcagctgagcaccgcaagcaatagccattgcggttggattccggaagaacgt |
| | ccgattagcagcattgcaggtcagatgagcgttgcatatattctggccgttcagctggttgatcagcagtgtctgctg |
| | agccagtttagcgaatttgatgataacctggaacgtccggaagtttgggatctggcacgtaaagttaccagcagcc |
| | agagcgaagaatttgatcaggatggtaattgtctgagcgcaggtcgtgttcgtattgaatttaatgatggttccagca |
| | ttaccgaaagcgttgaaaaaccgctgggtgttaaagaaccgatgccgaatgaacgtatcctgcataaatatcgtac |
| | cctggcaggtagcgttaccgatgaaagccgtgtgaaagaaattgaggatctggttctgggtctggatcgtctgacc |
| | gatattagtccgctgctggaactgctgaactgtccggttaaaagtccgctggtttaaactagtgaattcgcggccgc |
| | ataatgcttaagtcgaacagaaagtaatcgtattgtacacggccgcataatcgaaattaatacgactcactataggg |
| | gaattgtgagcggataacaattccccatcttagtatattagttaagtataagaaggagatatacatatggcagagctc |
| | actgtgactgaaagcaagaactccttcaatgctaagagcacccttgaagttggcgacaagtcctatgactacttcgc |
| | cctctctgcagtgcctggcatggagaagctgccgtactccctcaaggttctcggagagaaccttcttcgtaccgaa |
| | gacggcgcaaacatcaccaacgagcacattgaggctatcgccaactgggatgcatcttccgatccaagcatcga |
| | aatccagttcaccccagcccgtgttctcatgcaggacttcaccggtgtcccttgtgtagttgacctcgcaaccatgc |
| | gtgaggcagttgctgcactcggtggcgaccctaacgacgtcaacccactgaacccagccgagatggtcattgac |
| | cactccgtcatcgtggaggctttcggccgcccagatgcactggctaagaacgttgagatcgagtacgagcgcaa |
| | cgaggagcgttaccagttcctgcgttggggttccgagtccttctccaacttccgcgttgttcctccaggaaccggtat |
| | cgtccaccaggtcaacattgagtacttggctcgcgtcgtcttcgacaacgagggccttgcatacccagatacctgc |
| | atcggtaccgactcccacaccaccatggaaaacggcctgggcatcctgggctggggcgttggtggcattgaggc |
| | tgaagcagcaatgctcggccagccagtgtccatgctgatccctcgcgttgttggcttcaagttgaccggcgagatc |
| | ccagtaggcgttaccgcaactgacgttgtgctgaccatcaccgaaatgctgcgcgaccacggcgtcgtccagaa |
| | gttcgttgagttctacggctccggtgttaaggctgttccactggctaaccgtgcaaccatcggcaacatgtccccag |
| | agttcggctccacctgtgcgatgttcccaatcgacgaggagaccaccaagtacctgcgcctcaccggccgccca |
| | gaagagcaggttgcactggtcgaggcttacgccaaggcgcagggcatgtggctcgacgaggacaccgttgaag |
| | ctgagtactccgagtacctcgagctggacctgtccaccgttgttccttccatcgctggccctaagcgcccacagga |
| | ccgcatccttctctccgaggcaaaggagcagttccgtaaggatctgccaacctacaccgacgacgctgtttccgta |
| | gacacctccatccctgcaacccgcatggttaacgaaggtggcggacagcctgaaggcggcgtcgaagctgaca |
| | actacaacgcttcctgggctggctccggcgagtccttggctactggcgcagaaggacgtccttccaagccagtca |
| | ccgttgcatccccacagggtggcgagtacaccatcgaccacggcatggttgcaattgcatccatcacctcttgcac |
| | caacacctctaacccatccgtgatgatcggcgctggcctgatcgcacgtaaggcagcagaaaagggcctcaagt |
| | ccaagccttgggttaagaccatctgtgcaccaggttcccaggttgtcgacggctactaccagcgcgcagacctct |
| | ggaaggaccttgaggccatgggcttctacctctccggcttcggctgcaccacctgtattggtaactccggcccact |
| | gccagaggaaatctccgctgcgatcaacgagcacgacctgaccgcaaccgcagttttgtccggtaaccgtaactt |
| | cgagggacgtatctcccctgacgttaagatgaactacctggcatccccaatcatggtcattgcttacgcaatcgctg |
| | gcaccatggacttcgacttcgagaacgaagctcttggacaggaccaggacggcaacgacgtcttcctgaaggac |
| | atctggccttccaccgaggaaatcgaagacaccatccagcaggcaatctcccgtgagctttacgaagctgactac |
| | gcagatgtcttcaagggtgacaagcagtggcaggaactcgatgttcctaccggtgacaccttcgagtgggacga |
| | gaactccacctacatccgcaaggcaccttacttcgacggcatgcctgtcgagccagtggcagtcaccgacatcca |
| | gggcgcacgcgttctggctaagctcggcgactctgtcaccaccgaccacatctcccctgcttcctccattaagcca |
| | ggtacccctgcagctcagtacttggatgagcacggtgtggaacgccacgactacaactccctgggttccaggcgt |
| | ggtaaccacgaggtcatgatgcgcggcaccttcgccaacatccgcctccagaaccagctggttgacatcgcagg |
| | tggctacacccgcgacttcacccaggagggtgctccacaggcgttcatctacgacgcttccgtcaactacaaggc |
| | tgctggcattccgctggtcgtcttgggcggcaaggagtacggcaccggttcttcccgtgactgggcagctaaggg |
| | cactaacctgctcggaattcgcgcagttatcaccgagtccttcgagcgtattcaccgctccaacctcatcggtatgg |
| | gcgttgtcccactgcagttccctgcaggcgaatcccacgagtccctgggccttgacggcaccgagaccttcgaca |
| | tcaccggactgaccgcactcaacgagggcgagactcctaagactgtcaaggtcaccgcaaccaaggagaacg |
| | gcgacgtcgtcgagttcgacgcagttgtccgcatcgacaccccaggtgaggctgactactaccgccacggggc |
| | atcctgcagtacgtgctgcgtcagatggctgcttcttctaagtaaagatcttctggtaaagaaaccgctgctgcgaa |
| | atttgaacgccagcacatggactcgtctactagcgcagcttaattaacctaggctgctgccaccgctgagcaataa |
| | ctagcataaccccttggggcctctaaacgggtcttgaggggttttttgctgaaacctcaggcatttgagaagcacac |
| | ggtcacactgcttccggtagtcaataaaccggtaaaccagcaatagacataagcggctatttaacgaccctgccct |
| | gaaccgacgacaagctgacgaccgggtctccgcaagtggcacttttcggggaaatgtgcgcggaacccctattt |
| | gtttatttttctaaatacattcaaatatgtatccgctcatgaattaattcttagaaaaactcatcgagcatcaaatgaa |
| | actgcaatttattcatatcaggattatcaataccatatttttgaaaaagccgtttctgtaatgaaggagaaaactcacc |
| | gaggcagttccataggatggcaagatcctggtatcggtctgcgattccgactcgtccaacatcaatacaacctattaatt |
| | tcccctcgtcaaaaataaggttatcaagtgagaaatcaccatgagtgacgactgaatccggtgagaatggcaaaag |
| | tttatgcatttctttccagacttgttcaacaggccagccattacgctcgtcatcaaaatcactcgcatcaaccaaaccg |
| | ttattcattcgtgattgcgcctgagcgagacgaaatacgcggtcgctgttaaaaggacaattacaaacaggaatcg |
| | aatgcaaccggcgcaggaacactgccagcgcatcaacaatattttcacctgaatcaggatattcttctaatacctgg |
| | aatgctgttttcccggggatcgcagtggtgagtaaccatgcatcatcaggagtacggataaaatgcttgatggtcg |
| | gaagaggcataaattccgtcagccagtttagtctgaccatctcatctgtaacatcattggcaacgctacctttgccat |
| | gtttcagaaacaactctggcgcatcgggcttcccatacaatcgatagattgtcgcacctgattgcccgacattatcg |
| | cgagcccatttatacccatataaatcagcatccatgttggaatttaatcgcggcctagagcaagacgtttcccgttga |
| | atatggctcatactcttcctttttcaatattattgaagcatttatcagggttattgtctcatgagcggatacatatttg |
| | aatgtatttagaaaaataaacaaataggcatgcagcgctcttccgcttcctcgctcactgactcgctacgctcggtcgt |
| | tcgactgcggcgagcggtgtcagctcactcaaaagcggtaatacggttatccacagaatcaggggataaagccggaa |
| | agaacatgtgagcaaaaagcaaagcaccggaagaagccaacgccgcaggcgtttttccataggctccgccccc |
| | ctgacgagcatcacaaaaatcgacgctcaagccagaggtggcgaaacccgacaggactataaagataccaggc |
| | gtttccccctggaagctccctcgtgcgctctcctgttccgaccctgccgcttaccggatacctgtccgcctttctccct |
| | tcgggaagcgtggcgctttctcatagctcacgctgttggtatctcagttcggtgtaggtcgttcgctccaagctggg |
| | ctgtgtgcacgaaccccccgttcagcccgaccgctgcgccttatccggtaactatcgtcttgagtccaacccggta |
| | agacacgacttatcgccactggcagcagccattggtaactgatttagaggactttgtcttgaagttatgcacctgtta |
| | aggctaaactgaaagaacagattttggtgagtgcggtcctccaacccacttaccttggttcaaagagttggtagctc |
| | agcgaaccttgagaaaaccaccgttggtagcggtggtttttctttatttatgagatgatgaatcaatcggtctatcaag |
| | tcaacgaacagctattccgttactctagatttcagtgcaatttatctcttcaaatgtagcacctgaagtcagccccatac |
| | gatataagttgtaattctcatgttagtcatgccccgcgcccaccggaaggagctgactgggttgaaggctctcaag |
| | ggcatcggtcgagatcccggtgcctaatgagtgagctaacttacattaattgcgttgcgctcactgcccgctttcca |
| | gtcgggaaacctgtcgtgccagctgcattaatgaatcggccaacgcgcggggagaggcggtttgcgtattgggc |
| | gccagggtggtttttcttttcaccagtgagacgggcaacagctgattgcccttcaccgcctggccctgagagagttg |
| | cagcaagcggtccacgctggtttgccccagcaggcgaaaatcctgtttgatggtggttaacggcgggatataaca |
| | tgagctgtcttcggtatcgtcgtatcccactaccgagatgtccgcaccaacgcgcagcccggactcggtaatggc |
| | gcgcattgcgcccagcgccatctgatcgttggcaaccagcatcgcagtgggaacgatgccctcattcagcatttg |
| | catggtttgttgaaaaccggacatggcactccagtcgccttcccgttccgctatcggctgaatttgattgcgagtgag |
| | atatttatgccagccagccagacgcagacgcgccgagacagaacttaatgggcccgctaacagcgcgatttgct |
| | ggtgacccaatgcgaccagatgctccacgcccagtcgcgtaccgtcttcatgggagaaaataatactgttgatgg |
| | gtgtctggtcagagacatcaagaaataacgccggaacattagtgcaggcagcttccacagcaatggcatcctggt |
| | catccagcggatagttaatgatcagcccactgacgcgttgcgcgagaagattgtgcaccgccgctttacaggcttc |
| | gacgccgcttcgttctaccatcgacaccaccacgctggcacccagttgatcggcgcgagatttaatcgccgcgac |
| | aatttgcgacggcgcgtgcagggccagactggaggtggcaacgccaatcagcaacgactgtttgcccgccagtt |
| | gttgtgccacgcggttgggaatgtaattcagctccgccatcgccgcttccactttttcccgcgttttcgcagaaacgt |
| | ggctggcctggttcaccacgcgggaaacggtctgataagagacaccggcatactctgcgacatcgtataacgtta |
| | ctggtttcacattcaccaccctgaattgactctcttccgggcgctatcatgccataccgcgaaaggttttgcgccattc |
| | gatggtgtccgggatctcgacgctc |
| GroELS | atgaatattcgtccattgcatgatcgcgtgatcgtcaagcgtaaagaagttgaaactaaatctgctggcggcatcgtt |
| (SEQ ID NO: | ctgaccggctctgcagcggctaaatccacccgcggcgaagtgctggctgtcggcaatggccgtatccttgaaaat |
| 5) | ggcgaagtgaagccgctggatgtgaaagttggcgacatcgttattttcaacgatggctacggtgtgaaatctgaga |
| | agatcgacaatgaagaagtgttgatcatgtccgaaagcgacattctggcaattgttgaagcgtaatccgcgcacga |
| | cactgaacatacgaatttaaggaataaagataatggcagctaaagacgtaaaattcggtaacgacgctcgtgtgaa |
| | aatgctgcgcggcgtaaacgtactggcagatgcagtgaaagttaccctcggtccaaaaggccgtaacgtagttct |
| | ggataaatctttcggtgcaccgaccatcaccaaagatggtgtttccgttgctcgtgaaatcgaactggaagacaagt |
| | tcgaaaatatgggtgcgcagatggtgaaagaagttgcctctaaagcaaacgacgctgcaggcgacggtaccacc |
| | actgcaaccgtactggctcaggctatcatcactgaaggtctgaaagctgttgctgcgggcatgaacccgatggac |
| | ctgaaacgtggtatcgacaaagcggttaccgctgcagttgaagaactgaaagcgctgtccgtaccatgctctgact |
| | ctaaagcgattgctcaggttggtaccatctccgctaactccgacgaaaccgtaggtaaactgatcgctgaagcgat |
| | ggacaaagtcggtaaagaaggcgttatcaccgttgaagacggtaccggtctgcaggacgaactggacgtggttg |
| | aaggtatgcagttcgaccgtggctacctgtctccttacttcatcaacaagccggaaactggcgcagtagaactgga |
| | aagcccgttcatcctgctggctgacaagaaaatctccaacatccgcgaaatgctgccggttctggaagctgttgcc |
| | aaagcaggcaaaccgctgctgatcatcgctgaagatgtagaaggcgaagcgctggcaactctggttgttaacacc |
| | atgcgtggcatcgtgaaagtcgctgcggttaaagcaccgggcttcggcgatcgtcgtaaagctatgctgcaggat |
| | atcgcaaccctgactggcggtaccgtgatctctgaagagatcggtatggagctggaaaaagcaaccctggaaga |
| | cctgggtcaggctaaacgtgttgtgatcaacaaagacaccaccactatcatcgatggcgtgggtgaagaagctgc |
| | aatccagggccgtgttgctcagatccgtcagcagattgaagaagcaacttctgactacgaccgtgaaaaactgca |
| | ggaacgcgtagcgaaactggcaggcggcgttgcagttatcaaagtgggtgctgctaccgaagttgaaatgaaag |
| | agaaaaaagcacgcgttgaagatgccctgcacgcgacccgtgctgcggtagaagaaggcgtggttgctggtgg |
| | tggtgttgcgctgatccgcgtagcgtctaaactggctgacctgcgtggtcagaacgaagaccagaacgtgggtat |
| | caaagttgcactgcgtgcaatggaagctccgctgcgtcagatcgtattgaactgcggcgaagaaccgtctgttgtt |
| | gctaacaccgttaaaggcggcgacggcaactacggttacaacgcagcaaccgaagaatacggcaacatgatcg |
| | acatgggtatcctggatccaaccaaagtaactcgttctgctctgcagtacgcagcttctgtggctggcctgatgatc |
| | accaccgaatgcatggttaccgacctgccgaaaaacgatgcagctgacttaggcgctgctggcggtatgggcgg |
| | catgggtggcatgggcggcatgatgtaatga |
| pHCC-AtEcN | tcccttatgcgactcctgcattaggaaattaatacgactcactataggggaattgtgagcggataacaattcccctgt |
| (SEQ ID NO: | agaaataattttgtttaactttaataaggagatataccatgcaagcttatgaccaaacagagcgcagatagcaatgca |
| 6) | aaaagcggtgttaccagcgaaatttgtcattgggcaagcaatctggcaaccgatgatattccgagtgatgttctgga |
| | acgtgccaaatatctgattctggatggtattgcatgtgcatgggttggtgcacgtgttccgtggtcagaaaaatatgtt |
| | caggcaaccatgagctttgaaccgcctggtgcatgtcgtgttattggttatggccagaaactgggtccggttgcag |
| | cagcaatgaccaatagcgcatttattcaggccaccgaactggatgattaccatagcgaagcaccgctgcatagcg |
| | caagcattgttctgcctgccgtttttgcagcaagcgaagttctggcagaacagggtaaaaccattagcggtattgat |
| | gttattctggcagccattgttggttttgaaagcggtccgcgtattggtaaagcaatttatggtagcgatctgctgaata |
| | atggttggcattgtggtgcagtttatggtgcaccggcaggcgcactggccaccggcaaactgctgggtctgacac |
| | cggatagcatggaagatgcactgggtattgcctgtacccaggcatgtggtctgatgagcgcacagtatggtggtat |
| | ggttaaacgtgttcagcatggttttgcagcccgtaatggtctgctgggtggcctgctggcacatggtggttatgaag |
| | caatgaaaggtgtgctggaacgtagctatggtggttttctgaaaatgtttaccaaaggcaatggtcgtgaacctccg |
| | tataaagaagaagaagttgttgcaggtctgggtagcttttggcatacctttaccattcgcattaaactgtatgcatgttg |
| | tggtctggttcatggtccggtggaagcaattgaaaatctgcaaggtcgttatccggaactgctgaatcgtgcaaatc |
| | tgagcaatattcgtcatgttcatgttcagctgagcaccgcaagcaatagccattgcggttggattccggaagaacgt |
| | ccgattagcagcattgcaggtcagatgagcgttgcatatattctggccgttcagctggttgatcagcagtgtctgctg |
| | agccagtttagcgaatttgatgataacctggaacgtccggaagtttgggatctggcacgtaaagttaccagcagcc |
| | agagcgaagaatttgatcaggatggtaattgtctgagcgcaggtcgtgttcgtattgaatttaatgatggttccagca |
| | ttaccgaaagcgttgaaaaaccgctgggtgttaaagaaccgatgccgaatgaacgtatcctgcataaatatcgtac |
| | cctggcaggtagcgttaccgatgaaagccgtgtgaaagaaattgaggatctggttctgggtctggatcgtctgacc |
| | gatattagtccgctgctggaactgctgaactgtccggttaaaagtccgctggtttaaactagtgaattcgcggccgc |
| | ataatgcttaagtcgaacagaaagtaatcgtattgtacacggccgcataatcgaaattaatacgactcactataggg |
| | gaattgtgagcggataacaattccccatcttagtatattagttaagtataagaaggagatatacatatgatgtcgtcaa |
| | ccctacgagaagccagtaaagacacgttgcaggccaaagataaaacttaccactactacagcctgccgcttgctg |
| | ccaaatcactgggcgatatcacccgtctacccaagtcactcaaagttttgctcgaaaacctgctgcgttggcaggat |
| | ggtaaatcggttaccgaagaggatatccacgcgctggcgggatggctgaaaaatgcccatgctgaccgtgaaatt |
| | gcctaccgcccggcaagagtgctgatgcaggactttaccggcgtacctgccgttgttgatctggcggcaatgcgc |
| | gaagcggttaaacgcctcggcggcgatactgcaaaggttaacccactctcaccggtcgacctggtcattgaccac |
| | tcggtgaccgtcgatcgttttggtgatgatgaggcatttgaagaaaacgtacgcctggaaatggagcgcaaccac |
| | gaacgttatgtgttcctgaaatggggaaagcaggcgttcagtcggtttagcgtcgtgccgccaggcacaggcattt |
| | gccatcaggttaacctcgaatatctcggcaaagcagtgtggagcgaattgcaggacggtgaatggattgcttatcc |
| | ggatacactcgttggtactgactcgcacaccaccatgatcaacggccttggcgtgctggggggggcgttggtgg |
| | catcgaagcagaagccgcaatgttaggccagccggtttccatgcttatcccggacgtagtgggctttaaacttacc |
| | ggaaaattacgtgaaggtattaccgccacagacctggttctcactgttacccaaatgttgcgcaaacatggcgtggt |
| | ggggaaattcgtcgaattttatggtgatgggctggattcactaccgctggcggatcgcgccaccattgccaatatgt |
| | cgccagaatatggtgccacctgtggcttcttcccaatcgatgctgtcaccctcgattacatgcgtttaagcgggcgc |
| | agtgaagatcaggtcgagttggtcgaaaaatatgccaaagcgcagggcatgtggcgtaatccgggcgatgaacc |
| | aatttttaccagtgtgttagaactggatatgaatgacgttgaagcgagcctggcagggccaaaacgcccacaggat |
| | cgcgttgcactgcccgatgtaccaaaagcatttgctgccagtagcgaactggaagtgaatgccacgcataaagat |
| | cgcctgccggtcgattatgttatgaacggacatcagtatcagttacctgatggcgctgtggtcattgctgcgataac |
| | gtcgtgcaccaacacctctaacccaagtgtgttgatggccgcaggcttgctggcgaaaaaagccgtaacgctgg |
| | gcctcaagcggcaaccgtgggtcaaagcgtcgctggcaccgggttcgaaagtcgtttctgattatctggcaaaag |
| | cgaaactgacaccgtatctcgatgaactgggatttaaccttgtgggatacggttgtaccacctgtattggtaactctg |
| | gaccgttgcccgatcctatcgaaacggcaatcaaaaaaggcgatttaaccgtcggtgcagtgctgtccggcaacc |
| | gtaactttgaaggccgtatccatccgctggttaaaaccaactggctggcctcgccgccgctggtggttgcctatgc |
| | gctggcgggaaatatgaatatcaacctggcttctgagcctatcggccatgatcgcaaaggcgagccggtatatctg |
| | aaagatatctggccatcggcacaagaaattgcccgtgcggtagaccaagtctccacagaaatgttccgcaaagag |
| | tacgcagaagtttttgaaggcacatctgagtggaaggaaattaacgtcacacgatccgatacctacggttggcagg |
| | aggattcaacctatattcgcttatcgcctttctttgatgaaatgcaggcaacaccagcacctgtggaagatattcacg |
| | gtgcgcggatcctcgcaatgctgggggattcagtcaccactgaccatatctctccggcgggcagtattaagcccg |
| | acagcccggcgggtcgatatctacaaggtcggggtgttgagcggaaagactttaactcctatggttcgcggcgtg |
| | gtaaccatgaagtgatgatgcgcggcaccttcgccaatattcgcatccgtaatgaaatggtgcctggcgttgaagg |
| | ggggatgacgcgccatttacctgacagcgatgtcgtctctatttatgatgccgcaatgcgctataagcaggagcaa |
| | acgccgctggcggtgattgccgggaaagagtatggatcaggctccagccgtgactgggcggcaaaaggtccgc |
| | gtctgcttggtattcgtgttgtgattgccgaatcgtttgaacgaattcaccgttcgaatttaattggcatgggcatcctg |
| | ccgctggaatttccacaaggtgtgacgcgtaaaacgttagagcttaccggggaagagaagattgatattggtgatc |
| | tgcaaaatctacaacccggcgcgacggttccggtgacgcttacgcgcgcggatggtagccaggaagtcgtaccc |
| | tgccgttgtcgtatcgacaccgcgacggagttgacctactaccagaacgacggcattttgcattatgtcattcgtaat |
| | atgttgaagtaacaactatttgcttgccgggagctcggatctactagtctgcaggatctcgagtctggtaaagaaac |
| | cgctgctgcgaaatttgaacgccagcacatggactcgtctactagcgcagcttaattaacctaggctgctgccacc |
| | gctgagcaataactagcataaccccttggggcctctaaacgggtcttgaggggttttttgctgaaacctcaggcattt |
| | gagaagcacacggtcacactgcttccggtagtcaataaaccggtaaaccagcaatagacataagcggctatttaa |
| | cgaccctgccctgaaccgacgacaagctgacgaccgggtctccgcaagtggcacttttcggggaaatgtgcgcg |
| | gaacccctatttgtttatttttctaaatacattcaaatatgtatccgctcatgaattaattcttagaaaaactcatcga |
| | gcatcaaatgaaactgcaatttattcatatcaggattatcaataccatatttttgaaaaagccgtttctgtaatgaagg |
| | agaaaactcaccgaggcagttccataggatggcaagatcctggtatcggtctgcgattccgactcgtccaacatcaata |
| | caacctattaatttcccctcgtcaaaaataaggttatcaagtgagaaatcaccatgagtgacgactgaatccggtgaga |
| | atggcaaaagtttatgcatttctttccagacttgttcaacaggccagccattacgctcgtcatcaaaatcactcgcatc |
| | aaccaaaccgttattcattcgtgattgcgcctgagcgagacgaaatacgcggtcgctgttaaaaggacaattacaa |
| | acaggaatcgaatgcaaccggcgcaggaacactgccagcgcatcaacaatattttcacctgaatcaggatattctt |
| | ctaatacctggaatgctgttttcccggggatcgcagtggtgagtaaccatgcatcatcaggagtacggataaaatg |
| | cttgatggtcggaagaggcataaattccgtcagccagtttagtctgaccatctcatctgtaacatcattggcaacgct |
| | acctttgccatgtttcagaaacaactctggcgcatcgggcttcccatacaatcgatagattgtcgcacctgattgccc |
| | gacattatcgcgagcccatttatacccatataaatcagcatccatgttggaatttaatcgcggcctagagcaagacgt |
| | ttcccgttgaatatggctcatactcttcctttttcaatattattgaagcatttatcagggttattgtctcatgagcgga |
| | tacatatttgaatgtatttagaaaaataaacaaataggcatgcagcgctcttccgcttcctcgctcactgactcgctac |
| | gctcggtcgttcgactgcggcgagcggtgtcagctcactcaaaagcggtaatacggttatccacagaatcaggggataa |
| | agccggaaagaacatgtgagcaaaaagcaaagcaccggaagaagccaacgccgcaggcgtttttccataggct |
| | ccgcccccctgacgagcatcacaaaaatcgacgctcaagccagaggtggcgaaacccgacaggactataaag |
| | ataccaggcgtttccccctggaagctccctcgtgcgctctcctgttccgaccctgccgcttaccggatacctgtccg |
| | cctttctcccttcgggaagcgtggcgctttctcatagctcacgctgttggtatctcagttcggtgtaggtcgttcgctc |
| | caagctgggctgtgtgcacgaaccccccgttcagcccgaccgctgcgccttatccggtaactatcgtcttgagtcc |
| | aacccggtaagacacgacttatcgccactggcagcagccattggtaactgatttagaggactttgtcttgaagttat |
| | gcacctgttaaggctaaactgaaagaacagattttggtgagtgcggtcctccaacccacttaccttggttcaaagag |
| | ttggtagctcagcgaaccttgagaaaaccaccgttggtagcggtggtttttctttatttatgagatgatgaatcaatcg |
| | gtctatcaagtcaacgaacagctattccgttactctagatttcagtgcaatttatctcttcaaatgtagcacctgaagtc |
| | agccccatacgatataagttgtaattctcatgttagtcatgccccgcgcccaccggaaggagctgactgggttgaa |
| | ggctctcaagggcatcggtcgagatcccggtgcctaatgagtgagctaacttacattaattgcgttgcgctcactgc |
| | ccgctttccagtcgggaaacctgtcgtgccagctgcattaatgaatcggccaacgcgcggggagaggcggtttg |
| | cgtattgggcgccagggtggtttttttttcaccagtgagacgggcaacagctgattgcccttcaccgcctggccct |
| | gagagagttgcagcaagcggtccacgctggtttgccccagcaggcgaaaatcctgtttgatggtggttaacggcg |
| | ggatataacatgagctgtcttcggtatcgtcgtatcccactaccgagatgtccgcaccaacgcgcagcccggactc |
| | ggtaatggcgcgcattgcgcccagcgccatctgatcgttggcaaccagcatcgcagtgggaacgatgccctcatt |
| | cagcatttgcatggtttgttgaaaaccggacatggcactccagtcgccttcccgttccgctatcggctgaatttgattg |
| | cgagtgagatatttatgccagccagccagacgcagacgcgccgagacagaacttaatgggcccgctaacagcg |
| | cgatttgctggtgacccaatgcgaccagatgctccacgcccagtcgcgtaccgtcttcatgggagaaaataatact |
| | gttgatgggtgtctggtcagagacatcaagaaataacgccggaacattagtgcaggcagcttccacagcaatggc |
| | atcctggtcatccagcggatagttaatgatcagcccactgacgcgttgcgcgagaagattgtgcaccgccgcttta |
| | caggcttcgacgccgcttcgttctaccatcgacaccaccacgctggcacccagttgatcggcgcgagatttaatcg |
| | ccgcgacaatttgcgacggcgcgtgcagggccagactggaggtggcaacgccaatcagcaacgactgtttgcc |
| | cgccagttgttgtgccacgcggttgggaatgtaattcagctccgccatcgccgcttccactttttcccgcgttttcgca |
| | gaaacgtggctggcctggttcaccacgcgggaaacggtctgataagagacaccggcatactctgcgacatcgta |
| | taacgttactggtttcacattcaccaccctgaattgactctcttccgggcgctatcatgccataccgcgaaaggttttg |
| | cgccattcgatggtgtccgggatctcgacgctc |

The above Examples are used for illustration only but not for limiting the present disclosure. Modifications and alternations can be made to above Examples by anyone skilled in the art without departing from the scope of the present disclosure. Therefore, the range claimed by the present disclosure would be defined by attached Claims and encompassed within the disclosure of the present disclosure as long as that doesn't influence effects and purposes of the present disclosure.

### REFERENCES

1. Susin, M. F., Baldini, R. L., Gueiros-Filho, F., & Gomes, S. L. (2006). GroES/GroEL and DnaK/DnaJ have distinct roles in stress responses and during cell cycle progression in Caulobacter crescentus. Journal of bacteriology, 188(23), 8044-8053.
2. Hirata, H., Yamamura, I., Yasuda, K., Kobayashi, A., Tada, N., Suzuki, M., & Nagata, K. (1999). Separate cis-acting DNA elements control cell type-and tissue-specific expression of collagen binding molecular chaperone HSP47. Journal of Biological Chemistry, 274(50), 35703-35710.
3. Hao N, Chen Q, Dodd I B, Shearwin K E. ACS Synth Biol. 2021 Jun. 30. doi: 10.1021/acssynbio.1c00215. 10.1021/acssynbio.1c00215

1. A recombinant microorganism for production of itaconic acid and its derived monomers, comprising at least two genes located on a same expression vector, wherein the at least two genes comprise a gene encoding cis-aconitic acid decarboxylase and the other gene encoding aconitase, and the genome of the recombinant microorganism comprises a gene encoding a molecular chaperone protein GroELS.

2. The recombinant microorganism of claim 1, wherein the expression vector includes an inducible promoter gene for controlling expression of the at least two genes.

3. The recombinant microorganism of claim 2, wherein the inducible promoter is a T7 promoter.

4. The recombinant microorganism of claim 1, wherein the gene encoding cis-aconitic acid decarboxylase comprises a nucleotide sequence having at least 80% identity to SEQ ID NO: 1 and an activity identical to SEQ ID NO: 1.

5. The recombinant microorganism of claim 1, wherein the gene encoding aconitase comprises a nucleotide sequence having at least 80% identity to SEQ ID NO: 2 and an activity identical to SEQ ID NO: 2.

6. The recombinant microorganism of claim 1, wherein the gene encoding aconitase comprises a nucleotide sequence having at least 60% identity to SEQ ID NO: 3 and an activity identical to SEQ ID NO: 3.

7. The recombinant microorganism of claim 1, wherein the gene encoding the molecular chaperone protein GroELS comprises a nucleotide sequence having at least 80% identity to SEQ ID NO: 5 and an activity identical to SEQ ID NO: 5.

8. The recombinant microorganism of claim 1, which belongs to the genus of Escherichia, Klebsiella, Erwinia, Serratia, Providencia, Corynebacterium or Brevibacterium.

9. The recombinant microorganism of claim 8, which is a recombinant Escherichia coli BL21 (DE3) strain.

10. The recombinant microorganism of claim 9, which is deposited in the Deutsche Sammlung von Mikroorganismen und Zellkulturen (DSMZ) under the Accession No. DSM 34296.

11. The recombinant microorganism of claim 9, which is deposited in the Deutsche Sammlung von Mikroorganismen und Zellkulturen (DSMZ) under the Accession No. DSM 34297.

12. A method for production of itaconic acid and its derived monomers, comprising:
performing a reaction of the recombinant microorganism of claim 1 in a solution containing citric acid to convert the citric acid for producing itaconic acid and its derived monomers; and
isolating and obtaining the itaconic acid and its derived monomers from the solution.

13. The method of claim 12, wherein the solution is a mixed solution containing the citric acid and a citrate.

14. The method of claim 12, wherein the reaction is performed at a pH value between 5.0 and 7.0.

15. The method of claim 14, wherein the reaction is performed at a pH value between 5.0 and 6.0.

16. The method of claim 12, wherein the reaction is performed at a temperature between 22° C. and 37° C.

17. The method of claim 16, wherein the reaction is performed at a temperature between 22° C. and 30° C.
